(19) United States
(12) Patent Application Publication
(10) Pub. No.: US 2025/0078965 A1
(43) Pub. Date: Mar. 6, 2025

(54) Systems and Methods for Adjusting Covariates and Producing Treatment Effect Inferences in Randomized Controlled Trials

(71) Applicant: Unlearn.AI, Inc., San Francisco, CA (US)

(72) Inventors: Alyssa M. Vanderbeek, San Francisco, CA (US); Anna A. Vidovszky, Redwood City, CA (US); Jessica L. Ross, San Mateo, CA (US); Arman Sabbaghi, Martinez, CA (US); Jonathan R. Walsh, El Cerrito, CA (US); Charles Kenneth Fisher, Truckee, CA (US)

(73) Assignee: Unlearn.AI, Inc., San Francisco, CA (US)

(21) Appl. No.: 18/818,460

(22) Filed: Aug. 28, 2024

Related U.S. Application Data
(60) Provisional application No. 63/579,207, filed on Aug. 28, 2023.

(51) Int. Cl. G16H 10/20

(52) U.S. Cl. CPC G16H 10/20; G06N 7/00; G16B 40/00

(57) ABSTRACT

Systems and method for estimating treatment effects for a target trial in accordance with embodiments of the invention are illustrated. One embodiment includes a method. The method defines a skedastic function model, wherein defining the skedastic function model depends, at least in part, on target trial data. The method designs trial parameters for the target trial based in part on the skedastic function model. The method applies the trial parameters to a loss function to derive at least one minimizing coefficient, wherein a minimizing coefficient corresponds to a regression coefficient for an expected outcome to the target trial based on the trial parameters. The method computes standard errors for the at least one minimizing coefficient. The method quantifies, using the standard errors, values for uncertainty associated with the target trial. The method updates the trial parameters according to the uncertainty.

### CROSS-REFERENCE TO RELATED APPLICATIONS

The current application claims the benefit of and priority under 35 U.S.C. § 119(e) to U.S. Provisional Patent Application No. 63/579,207 entitled “A Weighted Prognostic Covariate Adjustment Method for Efficient and Powerful Treatment Effect Inferences in Randomized Controlled Trials,” filed Aug. 28, 2023. The disclosure of U.S. Provisional Patent Application No. 63/579,207 is hereby incorporated by reference in its entirety for all purposes.

### FIELD OF THE INVENTION

The present invention generally relates to prognostic models and, more specifically, the application of prognostic models to assessments of treatment effects.

### BACKGROUND

Randomized Controlled Trials (RCTs) are commonly used to assess the safety and efficacy of new treatments, including drugs and medical devices. In RCTs, subjects with particular characteristics are randomly assigned to one or more experimental groups receiving new treatments or to a control group receiving a comparative treatment (e.g., a placebo), and the outcomes from these groups are compared in order to assess the safety and efficacy of the new treatments.

Prognostic models are mathematical models that relate a subject's characteristics now to the risk of a particular future outcome, thereby allowing for RCTs to be efficiently represented. For example, Artificial Intelligence (AI) and Machine Learning (ML) algorithms may enable prognostic models to use historical data to create more efficient trials without introducing bias. When modelling RCTs in a medical context, prognostic models are used to compute prognostic scores, which correlate to the expected outcome or participants with specific pre-treatment covariates if they receive specific control treatments.

### SUMMARY OF THE INVENTION

Systems and techniques for applying prognostic models to assessment of experiment uncertainty are illustrated. One embodiment includes a method for estimating treatment effects for a target trial. The method defines a skedastic function model, wherein defining the skedastic function model depends, at least in part, on trial data that was applied in a trial. The method designs trial parameters for a target trial based in part on the skedastic function model. The method applies the trial parameters to a loss function to derive at least one minimizing outcome coefficient, wherein the at least one minimizing outcome coefficient corresponds to a regression coefficient for an expected outcome to the target trial based on the trial parameters. The method computes standard errors for the at least one minimizing outcome coefficient. The method quantifies, using the standard errors, values for uncertainty associated with the target trial. The method updates the trial parameters according to the uncertainty.

In a further embodiment, the standard errors are heteroskedasticity-consistent standard errors.

In another embodiment, the expected outcome is obtained through at least one of the group consisting of a digital twin and a prognostic model.

In another embodiment, defining the skedastic function model includes: calculating one or more predicted outcomes for the trial data; obtaining residuals corresponding to the one or more predicted outcomes for the trial data; and using the residuals to define the skedastic function model.

In further embodiment, predicted outcomes for the trial data are based on digital twin outputs.

In a further embodiment, the predicted outcomes are predictions from a regression model fitted on the trial data; and predictors of the regression model are means of the digital twin outputs.

In another further embodiment, the trial data includes participant data for an RCT.

In still another further embodiment, defining the skedastic function model further includes: applying parameters of the skedastic function model to a loss function for data from the target trial, to derive at least one minimizing model coefficient, wherein the at least one minimizing model coefficient includes a treatment effect coefficient; computing standard errors for the at least one minimizing model coefficient; calculating one or more predicted outcomes for the target trial; and defining the skedastic function model further based on variances corresponding to the one or more predicted outcomes for the target trial.

In a further embodiment, predicted outcomes for the target trial are based on digital twin outputs; and minimizing model coefficients are treatment effect coefficients.

In another embodiment, the loss function is a weighted least squares loss function.

In a further embodiment, at least one weight quantity of the weighted least squares loss function is inversely proportional to a predicted variance of outcomes of a participant in the target trial.

In another further embodiment, each weight quantity of the weighted least squares loss function has a positive value.

In yet another further embodiment, at least one weight quantity of the weighted least squares loss function is defined by: implementing, using trial data, an ordinary least squares fit; obtaining least squares coefficients from the ordinary least squares fit; and deriving, from the least squares coefficients and the trial parameters, the at least one weight quantity.

In another embodiment, updating the trial parameters according to the uncertainty includes determining a set of characteristics for the target trial, wherein the set of characteristics includes a number of subjects to be enrolled in each of a control arm and a treatment arm; and the uncertainty is based on at least one of a desired type-I error rate and a desired type-II error rate.

In yet another embodiment, updating the trial parameters includes at least one of: minimizing a total number of samples for at least one selected from the group consisting of a treatment arm of the target trial, a control arm of the target trial, and the target trial in totality; and performing a regression analysis based on the expected outcome.

In a further embodiment, an estimate for coefficients of the regression analysis is represented as: \hat{β}=(Z^{T}Z)⁻¹Z^{T}Y where Y is a vector corresponding to treatment outputs for each participant; and Z is a matrix for which each row (zᵢ) corresponds to a set of predictor variables for a participant (i).

In a further embodiment, the set of predictor variables for each participant include the expected outcome and a corresponding treatment for the participant.

In another further embodiment, minimizing a total number of samples is performed by deriving an expected variance reduction.

In a still further embodiment, deriving the expected variance reduction includes: obtaining a limit for the skedastic function model; deriving a set of estimated variance reductions for the previous trial, wherein the estimated variance reduction for each participant of the previous trial is derived from a ratio between a diagonal entry of a first matrix and a diagonal entry of a second matrix; and determining the expected variance reduction from the set of estimated variance reductions.

In yet another embodiment, Xᵢ is a vector of predictor variables for a participant i; and sᵢ² is a representation of the unknown outcome variance for the participant i. The first matrix is represented as:

$${Ω}_{1 / ℊ ( {σ}_{i}^{2} )}^{- 1} {Ω}_{{s}^{2} / {ℊ ( {σ}_{i}^{2} )}^{2}} {Ω}_{1 / ℊ ( {σ}_{i}^{2} )}^{- 1} ,$$

$$where :$$

$${Ω}_{1 / ℊ ( {σ}_{i}^{2} )} = E ( \frac{1}{ℊ ( {σ}_{i}^{2} )} {X}_{i} {X}_{i}^{T} ) , {Ω}_{{s}^{2} / {ℊ ( {σ}_{i}^{2} )}^{2}} = E ( \frac{{s}_{i}^{2}}{{ℊ ( {σ}_{i}^{2} )}^{2}} {X}_{i} {X}_{i}^{T} ) ,$$

and [symbol] (σᵢ²) is the limit of the skedastic function model for the participant i. The second matrix is represented as: Ω⁻¹Ωₛ_{²}Ω⁻¹, where: Ω=E(XᵢXᵢ^{T}), and Ωₛ_{²}=E(sᵢ²XᵢXᵢ^{T}).

Additional embodiments and features are set forth in part in the description that follows, and in part will become apparent to those skilled in the art upon examination of the specification or may be learned by the practice of the invention. A further understanding of the nature and advantages of the present invention may be realized by reference to the remaining portions of the specification and the drawings, which forms a part of this disclosure.

### BRIEF DESCRIPTION OF THE DRAWINGS

The description and claims will be more fully understood with reference to the following figures and data graphs, which are presented as exemplary embodiments of the invention and should not be construed as a complete recitation of the scope of the invention.

FIG. 1 illustrates uses for generative models in the analysis of clinical trials in accordance with various embodiments of the invention.

FIG. 2 illustrates using linear models and digital twins used to estimate treatment effects in accordance with several embodiments of the invention.

FIG. 3A illustrates a system for using generative models to estimate treatment effects in accordance with some embodiments of the invention.

FIG. 3B illustrates a system for borrowing information from digital twins to estimate treatment effects in accordance with many embodiments of the invention.

FIG. 4 conceptually illustrates an example of a process for designing a randomized controlled trial with prognostic effect estimation implemented in accordance with miscellaneous embodiments of the invention.

FIG. 5 conceptually illustrates a process for Fitted Function Weighted Prognostic Covariate Adjustment in accordance with numerous embodiments of the invention.

FIG. 6 discloses a process for defining skedastic function models in accordance with multiple embodiments of the invention.

FIG. 7 discloses a process for obtaining expected variance reductions in accordance with certain embodiments of the invention.

FIGS. 8A-8F disclose graphs illustrating demonstrated variance reduction assessments determined in accordance with many embodiments of the invention.

FIG. 9 illustrates a treatment analysis system that determines treatment effects in accordance with some embodiments of the invention.

FIG. 10 illustrates a treatment analysis element that executes instructions to perform processes that determine treatment effects in accordance with various embodiments of the invention.

FIG. 11 illustrates a treatment analysis application for determining treatment effects in accordance with numerous embodiments of the invention.

### DETAILED DESCRIPTION

Turning now to the drawings, systems and methods implemented in accordance with many embodiments of the invention may be implemented to yield efficient estimators and tests for treatment effects observed in randomized controlled trials (RCTs). In this disclosure, randomized controlled trials may also be referred to as experiments and randomized treatments. In obtaining treatment effect inferences from such methods, systems may be configured to incorporate predictions from generative artificial intelligence (AI) algorithms into aspects of RCT regression analyses, including but not limited to adjustment of covariates (i.e., independent variables) for regression analyses. By using generative AI, systems can effectively apply determined relationships (between the variation in the outcomes and at least some of the covariates) to produce outputs including but not limited to scalar features that can be optimized to explain variation in observed outcomes.

In accordance with some embodiments of the invention, training generative AI algorithms on historical control data (e.g., data obtained from previous RCTs) may enable construction of digital twin generators (DTGs). DTGs may correspond to entities including but not limited to RCT participants. DTGs configured in accordance with certain embodiments of the invention may utilize neural network architectures that can learn conditional generative models of patient trajectories (e.g., based on historical data). For example, DTGs may utilize the baseline covariates of participants (e.g., attributes observed prior to running RCTs) to generate probability distributions for potential control outcomes of the participants (digital twins). Summaries of the probability distribution from the DTGs may effectively predict trial outcomes. Additionally or alternatively, adjusting for covariates via regression can thus improve the quality of output including but not limited to treatment effect inferences, RCT designs, and/or decision rules for treatments.

Data sampled from generative models in accordance with a number embodiments of the invention may be referred to as ‘digital subjects’ throughout this description. In many embodiments, digital subjects can be generated to match given statistics of the treatment groups at the beginning of the study. Digital subjects in accordance with numerous embodiments of the invention can be generated for each subject in a study and the generated digital subjects can be used as digital twins for a counterfactual analysis. In various embodiments, generative models can be used to compute measures of response that are individualized to each patient and that can be used to assess the effects of the treatments. In many embodiments of the invention, estimates for treatment effects may be derived using linear regression models. Moreover, as suggested above, systems and methods in accordance with several embodiments of the invention can (additionally or alternatively) correct for bias that may be introduced by incorporating generated digital subject data.

Systems configured in accordance with multiple embodiments of the invention may endeavor to yield optimal treatment effect inferences in cases of homoskedasticity: i.e., consistency of outcome variance conditional on the covariates. Specifically, the case of nonconstant variance of outcomes (i.e., heteroskedasticity), adjustment methods may not satisfactorily improve treatment effect inferences. Systems and methods in accordance with several embodiments of the invention may enable (treatment effect-optimizing) adjustments with regard to various statistical terms including but not limited to standard errors. Covariate adjustments implemented in accordance with miscellaneous embodiments of the invention may involve regression models that include the covariates associated with the outcomes and/or the treatment (i.e., treatment indicator variable) as predictor variables.

In accordance with some embodiments, adjustments may be based on the incorporation of predictor variables including but not limited to prognostic scores determined by models of expected outcomes, and variances of the outcomes. In accordance with many embodiments of the invention, prognostic scores may refer to mean/average values of prognostic models including but not limited to digital twins. Additionally or alternatively, variances of outcomes may refer to variances of prognostic models including but not limited to digital twins, a specific classification of AI-based prognostic models. In this disclosure, digital twins may refer to digital representations of physical objects, processes, services, and/or environments with the capacity to behave like their counterparts in the real world. In the context of drug and medical studies, digital twins can take the form of representations of the range of potential control (placebo) outcomes of particular clinical trial participants given their baseline covariates/characteristics.

In accordance with many embodiments of the invention, adjustments (e.g., in the form of determined coefficients to the known and/or derived values) may be facilitated through methodology including but not limited to Prognostic Covariate Adjustment (PROCOVA) methods. PROCOVA methodology is disclosed in Unlearn.AI, Inc., “PROCOVA™ Handbook for the Target Trial Statistician.” Ver. 1.0, European Medicines Agency, incorporated herein by reference. In accordance with a number of embodiments of the invention PROCOVA methodology may be implemented using, but is not limited to weighted linear regression. Methods implementing such estimators may be referred to as “Weighted PROCOVA” in this disclosure.

In accordance with many embodiments of the invention, Weighted PROCOVA may adjust for moments of linear regression models, including but not limited to the mean and/or variance of the models, using information obtained from the DTGs. In some embodiments, the expectation and/or variance of probability distributions derived for individual participants may be used as covariates in modelling the mean and variance components for regression analyses. Such processes can improve RCT design by reducing the number of subjects required for different arms of the RCT. Processes in accordance with some embodiments of the invention can improve the ability of a system to accurately determine treatment effects from RCTs by increasing the statistical power of the trial. In many embodiments, the process of conducting RCTs can be improved from the design through the analysis and treatment decisions. Additionally or alternatively, processes may have impacts including but not limited to yield largely unbiased treatment effect estimators, reducing the variance of the treatment effect estimators, maintaining Type-I error rates, and/or increasing the power of the test for the treatment effects significantly. Moreover, the power boost of Weighted PROCOVA can be a direct function of its variance reduction.

Systems and methods implemented in accordance with many embodiments of the invention may carry multiple advantages. First, they can effectively summarize high-dimensional covariate vectors into scalars that can be highly correlated with the observed outcome (because the prognostic score is constructed from an AI algorithm optimized to predict the control outcome). Second, due to the AI algorithm(s) being trained on historical, out-of-sample control data, adjusting for the prognostic scores will not bias inferences, and/or decrease confidence interval coverage rates.

### I. Background

As mentioned above, systems and methods configured in accordance with various embodiments of the invention may be directed to determining the treatment effects of RCTs. RCT data can include panel data collected from subjects of RCTs and/or can be supplemented with generated subject data. Generated subject data in accordance with a number of embodiments of the invention can include (but are not limited to) digital subject data and/or digital twin data obtained from generative models.

### A. Generative Model Configurations

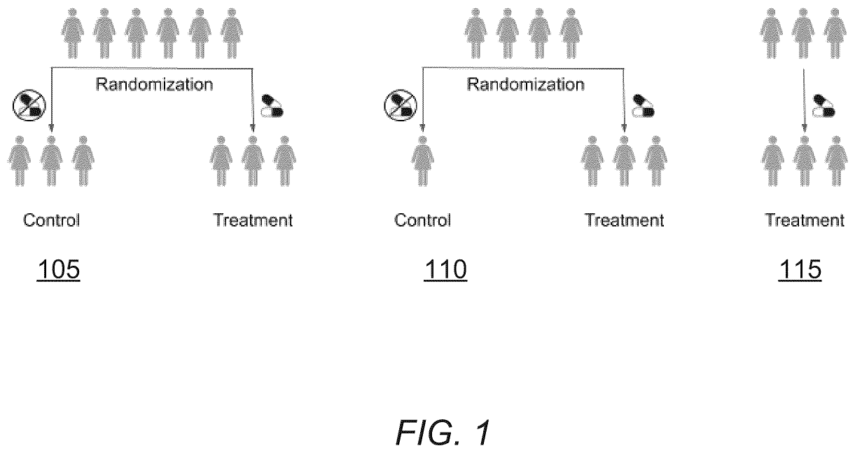

Examples of uses for generative models in the analysis of clinical trials in accordance with various embodiments of the invention are illustrated in FIG. 1. The first example 105 illustrates that generative models, digital subjects, and/or digital twins can be used to increase the statistical power of traditional randomized controlled trials. In the second example 110, generated data is used to decrease the number of subjects required to be enrolled in the control group of a randomized controlled trial. The third example 115 shows that generated data can be used in the external comparator arm of a single-arm trial.

In an RCT, a group of subjects with particular characteristics are randomly assigned to one or more experimental groups receiving new treatments and/or to a control group receiving a comparative treatment (e.g., a placebo), and the outcomes from these groups can be compared in order to assess the safety and efficacy of the new treatments. Without loss of generality, an RCT can be assumed to include i=1, . . . , N human subjects. These subjects are often randomly assigned to a control group and/or to a treatment group such that the probability of being assigned to the treatment group is the same for each subject regardless of any unobserved characteristics. The assignment of subject i to a group is represented by a treatment indicator variable wᵢ. For example, in a study with two groups wᵢ=0 if subject i is assigned to the control group and wᵢ=1 if subject i is assigned to the treatment group. The number of subjects assigned to the treatment group is N_{T}=Σᵢwᵢ and the number of subjects assigned to the control group is N_{C}=N−N_{T}.

In various embodiments, each subject i in an RCT can be described by a vector xᵢ(t) of covariate variables xᵢⱼ(t) at time t. In this description, the notation Xᵢ={xᵢ(t)}ₜ₌₁^{T} denotes the panel of data from subject i and x_{0,i} to denote the vector of data taken at time zero. An RCT is often concerned with estimating how a treatment affects an outcome yᵢ=ƒ(Xᵢ). The function ƒ(⋅) describes the combination of variables being used to assess the outcome of the treatment. Variables in accordance with a number of embodiments of the invention can include (but is not limited to) simple endpoints based on the value of a single variable at the end of the study, composite scores constructed from the characteristics of a patient at the end of the study, and/or time-dependent outcomes such as rates of range and/or survival times, among others. Approaches in accordance with various embodiments of the invention as described herein can be applied to analyze the effect of treatments on one or more outcomes (such as (but not limited to) those related to the efficacy and safety of the treatment).

Each subject has two potential outcomes. When the subject is assigned to the control group wᵢ=0, then y₁(0) would be the observed potential outcome. By contrast, when the subject is assigned to receive treatment wᵢ=1, then yᵢ(1) would be the observed potential outcome. In practice, subject may be assigned to one of the treatment arms such that the observed outcome is Yᵢ=yᵢ(0)(1−wᵢ)+wᵢyᵢ(1). Potential outcomes in accordance with many embodiments of the invention can include various measurements, such as, but not limited to conditional average treatment effect:

$$τ ( {x}_{0} ) = E [ w = 1 , {x}_{0} ] - E [ w = 0 , {x}_{0} ] \qquad ( i )$$

and/or the average treatment effect:

$$τ = E [ τ ( {x}_{0} ) ] = E [ y \vert w = 1 ] - E [ y \vert w = 0 ] . \qquad ( ii )$$

Processes in accordance with several embodiments of the invention can estimate these quantities with high accuracy and precision and/or can determine decision rules for declaring treatments to be effective that have low error rates.

It can be expensive, time-consuming and, in some cases, unethical to recruit human subjects to participate in RCTs. As a result, a number of methods have been developed for using external control arms to reduce the number of subjects required for an RCT. These methods typically fall into two buckets referred to as ‘historical borrowing’ and ‘external control’.

Historical borrowing refers to incorporating data from the control arms of previously completed trials into the analysis of a new trial. Typically, historical borrowing applies Bayesian methods using prior distributions derived from the historical dataset. Such methods can be used to increase the power of a randomized controlled trial, to decrease the size of the control arm, and/or even to replace the control arm with the historical data itself (i.e., an ‘external control arm’). Some examples of external control arms include control arms from previously completed clinical trials (also called historical control arms), patient registries, and data collected from patients undergoing routine care (called real-world data).

The design of RCTs to estimate the effect of new interventions on a given outcome can depend on various constraints, such as (but not limited to) the effect size one wishes to reliably detect, the power to detect that effect size, and/or the desired control of the type-I error rate. Of course, there may also be other considerations such as time and cost, and one may be interested in more than one particular outcome. Although many of the examples described herein are directed at optimizing for a single outcome, one skilled in the art will recognize that similar systems and methods can be used to optimize across multiple outcomes without departing from this invention.

Treatment effect estimators in accordance with many embodiments of the invention may presume a working model for observed outcomes Y=β₀+β₁w+β₂μ+ϵ where Y, w, and μ are a subject's outcome, treatment status, and prognostic score, respectively and ϵ is a noise term. In many embodiments of the invention, the noise term for participant i, may be determined such that ϵᵢ˜N(0, σᵢ²). In accordance with many embodiments of the invention model can be fit via ordinary least-squares and the resulting estimate of β₁, represented by \hat{β}₁, can be taken as the point estimate of the treatment effect. This estimate can be unbiased given treatment randomization without any assumptions about the veracity of the working linear model. Similarly, the assumption-free asymptotic sampling variance \hat{v}² ≡Var[\hat{β}₁] of this estimate is given by:

$${\hat{v}}^{2} = \frac{{\hat{σ}}_{0}^{2}}{{n}_{0}} + \frac{{\hat{σ}}_{1}^{2}}{{n}_{1}} - \frac{{n}_{0} {n}_{1}}{{n}_{0} + {n}_{1}} {( \frac{{\hat{ρ}}_{0} {\hat{σ}}_{0}}{{n}_{1}} + \frac{{\hat{ρ}}_{1} {\hat{σ}}_{1}}{{n}_{0}} )}^{2} \qquad ( iii )$$

$$in which {\hat{ρ}}_{w} = \frac{Cov [ {Y}_{w} , μ ]}{\sqrt{Var [ μ ] Var [ {Y}_{w} ]}}$$

(where Y_{w} denotes potential outcomes under treatment w=1 and control w=0, \hat{σ}_{w}²=Var[Y_{w}], n₀ and n₁ are the number of enrolled control and treated subjects).

An effect estimate can be declared to be “statistically significant” at level α if a p<α where p=2*(min {Φ(\hat{β}1/\hat{v}), (1−Φ(\hat{β}₁/\hat{v}))} is the two-sided p-value, \hat{v} is the standard error of \hat{β}₁, and Φ denotes the CDF of the standard normal density. The probability that p<α when, in reality, the treatment effect is β₁ is given by

$$Power = Φ ( {Φ}^{- 1} ( α / 2 ) + \frac{{β}_{1}}{v} ) + Φ ( {Φ}^{- 1} ( α / 2 ) - \frac{{β}_{1}}{v} ) . \qquad ( iv )$$

To power a trial to a given level (e.g. 80%) one must first estimate values for σ_{w}² and ρ_{w} using prior data (discussed below) or expert opinion. The power formula can then be composed with the variance formula with σ_{w}² and ρ_{w} fixed at their estimates \hat{σ}_{w}² and \hat{ρ}_{w}. The resulting function returns power for any values of n₀ and n₁.

The goal of a sample size calculation in the design of a clinical trial (e.g., that uses PROCOVA) can be to estimate n₀ and n₁ required to achieve the required power. However, one needs an additional constraint such as (but not limited to) a chosen randomization ratio n₀/n₁, or minimizing the total trial size n₀+n₁. In this example, the randomization ratio is pre-specified, but the same principles can be easily applied to other situations.

In numerous embodiments, processes for designing a trial can be based on a generative (or prognostic) model. Prognostic models in accordance with many embodiments of the invention can be trained (e.g., based on a prior trial) or pre-trained. Processes can then estimate the variances, σ_{w}² and correlations, ρ_{w} of the control arm of the trial. One method for obtaining these estimates is to use historical data, such as data from the placebo control arms of previous trials performed on similar populations. In numerous embodiments, estimates can be based on vectors of outcomes for these subjects, gathered during the trials, and their corresponding prognostic, calculated with the prognostic model from each subject's vector of baseline covariates.

In some embodiments, control-arm marginal outcome variance σ₀² can be estimated with the usual estimator

$${\hat{σ}}_{0}^{2} = \frac{1}{{n}^{′} - 1} \sum {( {Y}_{i}^{′} - {\overline{Y}}^{′} )}^{2}$$

where Y̅′ is the sample average. The correlation ρ₀ between μ′ and Y′ can be estimated by \hat{ρ}₀=Σ (Y′ᵢ−Y̅′)(μ′ᵢ−μ̅′)/\sqrt{Σ(Y′ᵢ−Y̅′)² Σ(μ′ᵢ−μ̅′)²}, the usual sample correlation coefficient. These values may be inflated (for σ₀²) or deflated (for ρ₀) in order to provide more conservative estimates of power.

In certain embodiments, an inflation parameter λ_{w}, for the variance and a deflation parameter γ_{w} for the correlation can be applied to sample size calculation. Inflation and deflation parameters can be used to account for the prognostic model. Define the target effect size β₁*, the significance threshold α, the desired power level ζ, fraction of subjects to be randomized to the active arm π, and dropout rate d. Define γ_{w}≥1 and λ_{w}∈ [0,1] for w=0, 1. Define the variance of the potential outcome under active treatment w in the planned trial as γ_{w}²\hat{σ}₀², so that a large γ_{w} inflates the estimated variance. Similarly, define the correlation between the potential outcome and the prognostic model under active treatment w as λ_{w}\hat{ρ}₀, so that a small λ_{w} deflates the estimated correlation. Then n could be minimized using a numerical optimization algorithm (such as a binary search) such that

$$ζ \geq Φ ( {Φ}^{- 1} ( \frac{α}{2} ) + \frac{{β}_{1}^{*}}{v} ) + Φ ( {Φ}^{- 1} ( \frac{α}{2} ) - \frac{{β}_{1}^{*}}{v} ) ,$$

$$with$$

$${v}^{2} = \frac{1}{n} ( \frac{{γ}_{0}^{2} {\hat{σ}}_{0}^{2}}{1 - π} + \frac{{γ}_{1}^{2} {\hat{σ}}_{0}^{2}}{π} + \frac{{\hat{θ}}^{2} - 2 {\hat{θ}}_{⋆} \hat{θ}}{π ( 1 - π )} ) , \hat{θ} = {\hat{ρ}}_{0} {\hat{σ}}_{0} ( ( 1 - π ) {λ}_{0} {γ}_{0} + π {λ}_{1} {γ}_{1} ) ,$$

$$and$$

$${\hat{θ}}_{⋆} = {\hat{ρ}}_{0} {\hat{σ}}_{0} ( π {λ}_{0} {γ}_{0} + ( 1 - π ) {λ}_{1} {γ}_{1} ) .$$

The minimum sample size can be estimated to be

$${n}_{d} = \frac{n}{1 - d} .$$

Unlike the variances and correlations for a control arm, the corresponding values for the treatment arm can rarely be estimated from data because treatment-arm data for the experimental treatment is likely to be scarce or unavailable. In many embodiments, processes can assume σ₀²=σ₁² and ρ₀=ρ₁, the latter of which holds exactly if the effect of treatment is constant across the population. It may also be prudent (and conservative) to assume a slightly higher value for σ₁² and a slightly smaller value for ρ₁ relative to their control-arm counterparts.

With the four parameters σ_{w}² and ρ_{w} specified, the power formula can be computationally optimized over n₀ and n₁ in the desired randomization ratio n₀/n₁ until the minimum values of n₀ and n₁ are found such that the output power meets or exceeds the desired value (e.g., with a numerical optimization scheme).

In many cases, a trial will aim to assess the effect of the intervention on many different outcomes. Processes in accordance with several embodiments of the invention can use multiple prognostic models (e.g., one to predict each outcome of interest) and/or multivariate prognostic models. Depending on the variances of the outcomes, and the accuracy with which they can be predicted, sample size calculations on the various outcomes of interest may suggest different required sample sizes. In this case, one could simply choose the smallest sample size that meets the minimum required statistical power on each of the outcomes of interest.

Systems configured in accordance with some embodiments apply machine learning methods to create simulated subject records. In addition to data from RCTs, generative models in accordance with several embodiments of the invention can link the baseline characteristics x₀ and the control potential outcomes y⁽⁰⁾ through joint probability distributions p_{θ}_{_{J}}(y⁽⁰⁾, x₀) and conditional probability distributions p_{θ}_{_{C}}(x₀), in which θ_{J}, and θ_{C} are the parameters of the joint and conditional distributions, respectively. Note that a model of the joint distribution will also provide a model of the conditional distribution, but the converse is not true.

In several embodiments, simulated subject records can be sampled from probabilistic generative models that can be trained on various data, such as (but not limited to) one or more of historical, registry, and/or real-world data. Such models can allow one to extrapolate to new patient populations and study designs.

In some embodiments, generative models may create data in a specialized format—either directly or indirectly—such as the Study Data Tabulation Model (SDTM) and/or the Neyman-Rubin Causal Model to facilitate seamless integration into standard workflows. In a variety of embodiments, generating entire panels of data can be attractive because many of the trial outcomes (such as primary, secondary, and exploratory endpoints as well as safety information) can be analyzed in a parsimonious way using a single generative model.

Systems and methods in accordance with numerous embodiments of the invention can provide various approaches for incorporating data from probabilistic generative models into the analysis of RCTs. In numerous embodiments, such methods can be viewed as borrowing from a model, as opposed to directly borrowing from a historical dataset. As generative models, from which data can be borrowed, may be biased (for example, due to incorrect modelling assumptions), systems and methods in accordance with a number of embodiments of the invention can account for these potential biases in the analysis of RCTs. Generative models in accordance with various embodiments of the invention can provide control over the characteristics of each simulated subject at the beginning of the study. For example, processes in accordance with various embodiments of the invention can create one or more digital twins for each human subject in the study. Processes in accordance with certain embodiments of the invention can incorporate digital twins to increase statistical power and can provide more individualized information than traditional study designs, such as study designs that borrow population-level information and/or that use nearest neighbor matches to patients in historical or real-world databases.

### B. Training Generative Models

In several embodiments, processes can receive historical data that can be used to pre-train generative models and/or to determine prior distribution for (e.g., Bayesian) analyses. Historical data in accordance with numerous embodiments of the invention can include (but is not limited to) control arms from historical control arms, patient registries, electronic health records, and/or real-world data.

Digital subject data may be generated using generative models. Generative models in accordance with certain embodiments of the invention can be trained to generate potential outcome data based on characteristics of an individual and/or a population. Digital subject data in accordance with several embodiments of the invention can include (but are not limited to) panel data, outcome data, etc. In numerous embodiments, generative models can be trained directly on a specific outcome p(y|x₀). For example, if a goal of using the generative model is to increase the statistical power for the primary analysis of a randomized controlled trial then it may be sufficient (but not necessary) to only use a model of p(y|x₀).

Alternatively, or conjunctively, generative models may be trained to generate panel data that can be used in the analysis of a clinical trial. Data for a subject in a clinical trial are typically a panel; that is, it describes the observed values of multiple characteristics at multiple discrete timepoints (e.g. visits to the clinical trial site). For example, if a goal of using the generative model is to reduce the number of subjects in the control group of the trial, or as an external comparator for a single-arm trial, then generated panel data in accordance with many embodiments of the invention can be used to perform many or all of the analyses of the trial.

In several embodiments, generative models can include (but are not limited to) traditional statistical models, generative adversarial networks, recurrent neural networks, Gaussian processes, autoencoders, autoregressive models, variational autoencoders, and/or other types of probabilistic generative models. For example, processes in accordance with several embodiments of the invention can use sequential models such as (but not limited to) Conditional Restricted Boltzmann Machines for the full joint distribution of the panel data, p(X), from which any outcome can be computed.

Systems and methods in accordance with numerous embodiments of the invention may determine treatment effects for RCTs using generated digital subject data. Generative models in accordance with many embodiments of the invention can be incorporated into the analysis of an RCT in a variety of different ways for various applications. In many embodiments, generative models can be used to estimate treatment effects by training separate generative models based on data from the control and treatment arms of previous trials. Processes in accordance with many embodiments of the invention can use generative models to generate digital subjects to supplement control arms in RCTs. In certain embodiments, processes can use generative models to generate digital twins for individuals in the control and/or treatment arms. Generative models in accordance with numerous embodiments of the invention may be used to define individualized responses to treatment. Various methods for determining treatment effects in accordance with various embodiments of the invention are described in greater detail herein.

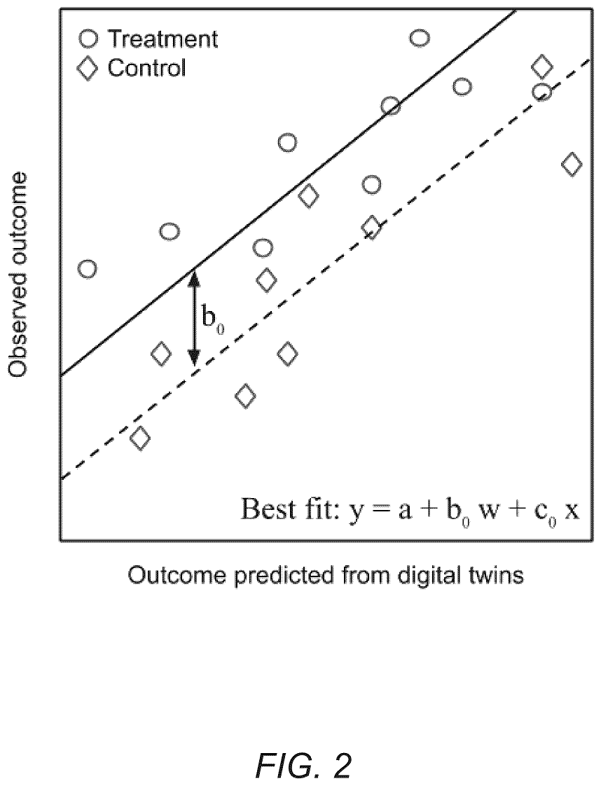

An example of using linear models and digital twins to estimate treatment effects in accordance with an embodiment of the invention is illustrated in FIG. 2. This drawing illustrates the concept using a simple analysis of a continuous outcome. The x-axis represents the prediction for the outcome from the digital twins, and the y-axis represents the observed outcome of the subjects in the RCT. A linear model is fit to the data from the RCT, adjusting for the outcome predicted from the digital twins. When no interactions are included, then two parallel lines are fit to the data: one to the control group and one to the treatment group. The distance between these lines is an estimate for the treatment effect. Both frequentist and Bayesian methods may be used to analyze the generalized linear model.

In several embodiments, treatment effects can be determined by fitting generalized linear models (GLMs) to the generated digital subject data and/or the RCT data. In a number of embodiments, multilevel GLMs can be set up so that the parameters (e.g., the treatment effect) can be estimated through maximum likelihood or Bayesian approaches. In a frequentist approach, one can test the null hypothesis β₀=0, whereas the Bayesian approach may focus on the posterior probability Prob(β₀≥0|data, prior).

In many embodiments, processes can estimate treatment effects by training two new generative models: a treatment model using the data from the treatment group, t,?, and a control model using the data from the control group, t,?. In a variety of embodiments, full panels of data from an RCT can be used to train generative models to create panels of generated data. Such processes can allow for the analysis of many outcomes (including (but not limited to) primary, secondary, and exploratory efficacy endpoints as well as safety information) by comparing the trained treatment models against trained control models. For simplicity, the notation p(y,x₀) will be used instead of p(X), with the understanding that the former can always be obtained from the latter by generating a panel of data X and then computing a specific outcome y=ƒ(X) from the panel.

In one embodiment, generative models for the control condition (e.g., a Conditional Restricted Boltzmann Machine) can be trained on historical data from previously completed clinical trials. Then, two new generative models for the control and treatment groups can be obtained by solving minimization problems:

$${min}_{{θ}_{{J}_{0}}} \{ - {\sum }_{i} ( 1 - {w}_{i} ) log {p}_{{θ}_{{J}_{0}}} ( {Y}_{i} , {x}_{0 , i} \vert {w}_{0} ) + {λ}_{0} D ( {p}_{{θ}_{{J}_{0}}} , {p}_{{θ}_{J}} ) \} \qquad ( vi )$$

$${min}_{{θ}_{{J}_{1}}} \{ - {\sum }_{i} {w}_{i} log {p}_{{θ}_{{J}_{1}}} ( {Y}_{i} , {x}_{0 , i} \vert {w}_{1} ) + {λ}_{1} D ( {p}_{{θ}_{{J}_{1}}} , {p}_{{θ}_{J}} ) \} \qquad ( vii )$$

in which λ₀ and λ₁ are prior parameters that describe how well pre-trained generative models describe the outcomes in the two arms of the RCT, and D(⋅,⋅) is a measure of the difference between two generative models such as (but not limited to) the Kullback-Leibler divergence.

In some embodiments, the new generative models may, additionally or alternatively, be conditional generative models (e.g., Conditional Restricted Boltzmann Machines). The estimate for the treatment effect can then be computed as:

$$\hat{τ} = \int dy {dxyp}_{{θ}_{{J}_{1}}} ( y , x \vert {w}_{1} ) - \int dy {dxyp}_{{θ}_{{J}_{0}}} ( y , x \vert {w}_{0} ) . \qquad ( viii )$$

In several embodiments, treatment effects can be computed by drawing samples from the control and treatment models and comparing the distributions of the samples. Processes in accordance with some embodiments of the invention can further tune the computation of treatment effects by adjusting for the uncertainty in treatment effect estimates. In several embodiments, the uncertainty in treatment effect estimates (σ_{\hat{τ}}) can be obtained using bootstraps by repeatedly resampling the data from the RCTs (with replacement), training the updated generative models, and/or computing estimates for the treatment effects (wherein the uncertainty is the standard deviation of these estimates). In a number of embodiments, point estimates for the treatment effect and the estimates for uncertainty can be used to perform hypothesis tests in order to create decision rules.

In numerous embodiments, processes can begin with distributions π(θ_{J}) for the parameters of the generative model (e.g., obtained from a Bayesian analysis of historical data). Then, posterior distributions for θ_{J}_{₀} and θ_{J}_{₁} can be estimated by applying Bayes rule:

$$log π ( {θ}_{{J}_{0}} ) = constant + {\sum }_{i} ( 1 - {w}_{i} ) {p}_{{θ}_{{J}_{0}}} ( {Y}_{i} , {x}_{i} \vert {w}_{0} ) + {λ}_{0} log π ( {θ}_{J} ) \qquad ( ix )$$

$$log π ( {θ}_{{J}_{1}} ) = constant + {\sum }_{i} {w}_{i} {p}_{{θ}_{{J}_{1}}} ( {Y}_{i} , {x}_{i} \vert {w}_{1} ) + {λ}_{1} log π ( {θ}_{J} ) .$$

In certain embodiments, point estimates for the treatment effect can be calculated as the mean of the posterior distribution

$$\hat{τ} = \int dy dxd {θ}_{{J}_{1}} y {p}_{{θ}_{{J}_{1}}} ( y , x \vert {w}_{1} ) π ( {θ}_{{J}_{1}} ) - \int dy dxd {θ}_{{J}_{0}} {yp}_{{θ}_{{J}_{0}}} ( y , x \vert {w}_{0} ) π ( {θ}_{{J}_{1}} ) , \qquad ( x )$$

where the uncertainty is the variance of the posterior distribution

$${δ}^{2} τ = \int dy dxd {θ}_{{J}_{1}} {y}^{2} {p}_{{θ}_{{J}_{1}}} ( y , x \vert {w}_{1} ) π ( {θ}_{{J}_{1}} ) - \int dy dxd {θ}_{{J}_{0}} {y}^{2} {p}_{{θ}_{{J}_{0}}} ( y , x \vert {w}_{0} ) π ( {θ}_{{J}_{1}} ) - {\hat{τ}}^{2} . \qquad ( xi )$$

As above, point estimates for the treatment effect and estimates for their uncertainty can be used to perform a hypothesis test in order to create a decision rule in accordance with certain embodiments of the invention. Processes in accordance with a variety of embodiments of the invention can train conditional generative models t,? and t,?, as opposed to (or in conjunction with) joint generative models, in order to estimate treatment effects that are conditioned on the baseline covariates x₀.

It can be difficult to determine the operating characteristics of decision rules based on these methods. Specifically, extensive simulations can be required in order to estimate the type-I error rate (i.e., the probability that an ineffective treatment would be declared to be effective) and/or the type-II error rate (i.e., the probability that an effective treatment would be declared ineffective). Well-characterized operating characteristics are required for many applications of RCTs and, as a result, this approach is often impractical. Generative models that rely on modern machine learning techniques are typically computationally expensive to train. As a result, using the bootstrap and/or Bayesian methods to obtain uncertainties required to formulate reasonable decision rules can be quite challenging.

### C. Applying Generative Models

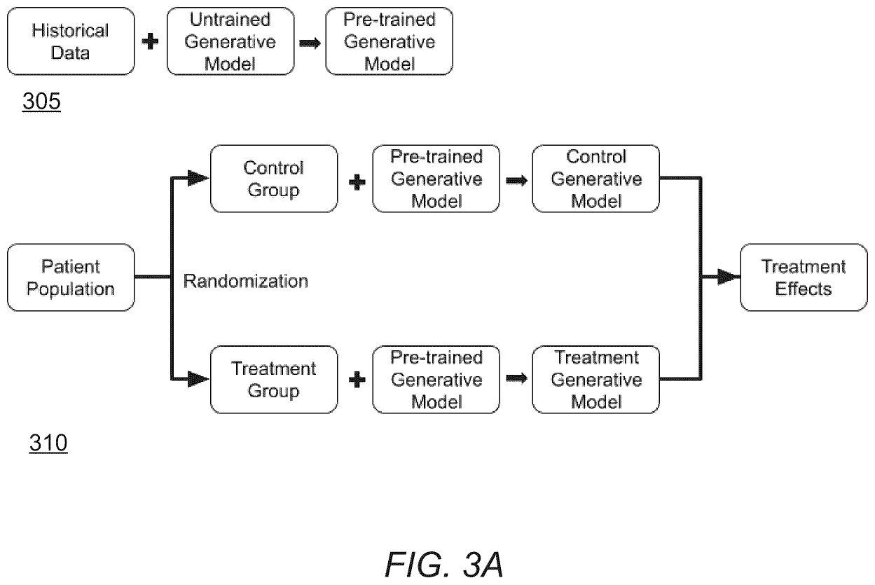

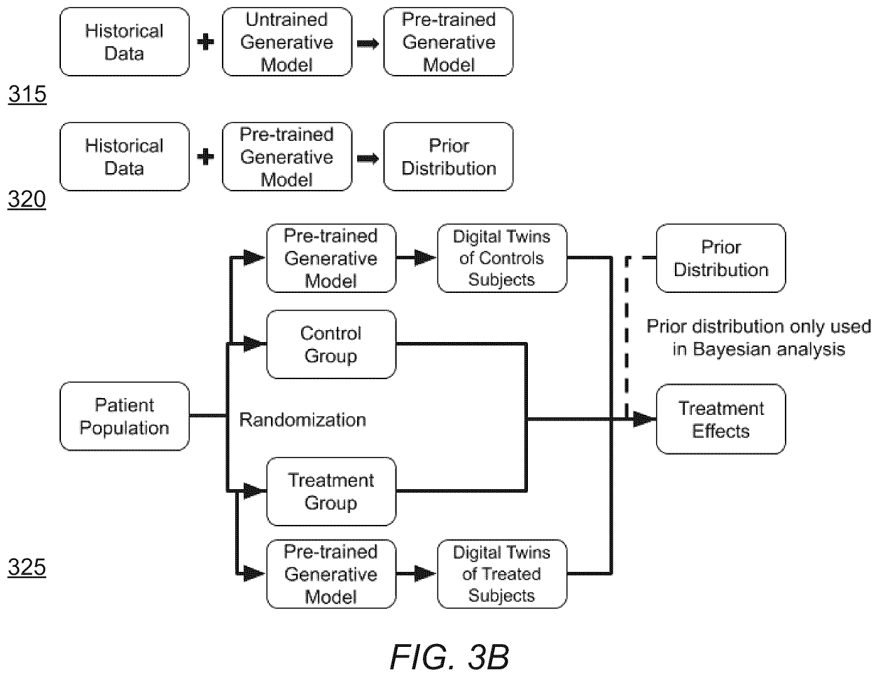

An example of using generative models to estimate treatment effects in accordance with an embodiment of the invention is illustrated in FIG. 3A. In the first stage 305, an untrained generative model of the control condition is trained using historical data, such as (but not limited to), data from previously completed clinical trials, electronic health records, and/or other studies. In the second stage 310, a patient population is randomly divided into a control group and a treatment group as part of a randomized controlled trial. Patients from the population can be randomized into the control and treatment groups with unequal randomization in accordance with a variety of embodiments of the invention. In this example, two new generative models are trained: one for the control group and one for the treatment group. In certain embodiments, control and treatment generative models can be based on a pre-trained generative model but can be additionally trained to reflect new information from the RCT. Outputs from the control and generative models can then be compared to compute the treatment effects. In several embodiments, Bayesian methods and/or bootstrap may be used to estimate uncertainties in the treatment effects and decision rules based on p-values and/or posterior probabilities may be applied.

Some methods estimate treatment effects using GLMs while adjusting for covariates. For example, one may perform a regression of the final outcome in the trial against the treatment indicator and a measure of disease severity at the start of the trial. As long as the covariate was measured before the treatment was assigned in a randomized controlled trial, then adjusting for the covariate will not bias the estimate for the treatment effect in a frequentist analysis. When using covariate adjustment, the statistical power is a function of the correlation between the outcome and the covariate being adjusted for; the larger the correlation, the higher the power.

In theory, the covariate that is most correlated with the outcome that one could obtain is an accurate prediction of the outcome. Therefore, another method to incorporate generative models into RCTs in accordance with a variety of embodiments of the invention is to use generative models to predict outcomes and to adjust for the predicted outcomes in a GLM for estimating the treatment effect. Let Eₚ[yᵢ] and Varₚ[yᵢ] denote the expected value and variance of the outcome predicted for subject i by the generative model, respectively. Depending on the type of generative model, these moments may be computable analytically or, more generally, by drawing samples from the generative model p(x_{0,i}) and computing Monte Carlo estimates of the moments in accordance with a number of embodiments of the invention. The number of samples used to compute the Monte Carlo estimates can be a parameter selected by the researcher. As above, processes in accordance with several embodiments of the invention can use generative models that generate panel data so that a single generative model may be used for analyses of many outcomes in a given trial (e.g., primary, secondary, and exploratory endpoints as well as safety information). In a number of embodiments, rather than predictions for given outcomes, predictions of multiple outcomes derived from a generative model may all be included in a GLM for particular outcomes. Samples drawn from the generative models in accordance with several embodiments of the invention can be conditioned on the characteristics of subjects at the start of the trial, also referred to as digital twins of that subject.

In many embodiments, digital twins can be incorporated into an RCT in order to estimate the treatment effect by fitting a GLM of the form:

$$( xii )$$

$$g ( E [ {y}_{i} ] ) = a + ( {b}_{0} + {\sum }_{j} {b}_{j} {x}_{0 , ij} ) {w}_{i} + ( {c}_{0} + {\sum }_{j} {c}_{j} {x}_{0 , ij} ) g ( {E}_{p} [ {y}_{i} ] ) + {\sum }_{j} {d}_{j} {x}_{ij} + ( {z}_{0} + {\sum }_{j} {z}_{j} {x}_{0 , ij} ) {w}_{i} g ( {E}_{p} [ {y}_{i} ] )$$

in which g(⋅) is a link function. For example, g(μ)=μ corresponds to a linear regression and g(μ)=log(μ/(1−μ)) corresponds to logistic regression. This framework in accordance with numerous embodiments of the invention can also include Cox proportional hazards models used for survival analysis as a special case. In many embodiments, some of these coefficients may be set to zero to create simpler models. The above equation can be generalized to various applications and implementations. The terms involving the b coefficients represent the treatment effect, which may depend on the baseline covariates x₀. The terms involving the c coefficients represent potential bias in the generative model, which may depend on the baseline covariates x₀. The terms involving the d coefficients represent potential baseline differences between the treatment and control groups in the trial. The terms involving the z coefficients reflect that the relationship between the predicted and observed outcomes may be affected by the treatment. The model can be fit using any of a variety of methods for fitting GLMs. One skilled in the art will recognize that it is trivial to include other predictions from the generative model as covariates if desired.

In accordance with many embodiments of the invention, uncertainty values can be applied in a variety of ways. They may be estimated analytically. Alternatively, or conjunctively, processes in accordance with many embodiments of the invention can estimate uncertainties using bootstraps by repeatedly resampling the data (with replacement) and re-fitting the model; the uncertainties can be the standard deviations of the coefficients computed by this resampling procedure. In some embodiments, point estimates for the treatment effect and estimates for their uncertainty can be used to perform a hypothesis test in order to create a decision rule.

In some embodiments, variances of the outcomes can be modeled through another GLM that adjusts for the variance of the outcome that is predicted by the generative model. For example, variances in accordance with many embodiments of the invention can be modeled as follows:

$$( xiii )$$

$$G ( Var [ {y}_{i} ] ) = α + ( {β}_{0} + {\sum }_{j} {β}_{j} {x}_{0 , ij} ) {w}_{i} + ( {γ}_{0} + {\sum }_{j} {γ}_{j} {x}_{0 , ij} ) G ( {Var}_{p} [ {y}_{i} ] ) + {\sum }_{j} {δ}_{j} {x}_{ij} + ( {ζ}_{0} + {\sum }_{j} {ζ}_{j} {x}_{0 , ij} ) {w}_{i} G ( {Var}_{p} [ {y}_{i} ] )$$

in which G(⋅) is a link function that is appropriate for the variance. For example, G(σ²)=log(σ²) can be used for a continuous outcome. In many embodiments, some of these coefficients may be set to zero to create simpler models. One skilled in the art will recognize that other predictions from the generative model can be included as covariates when desired.

Well-trained generative models in accordance with certain embodiments of the invention can have g(E[yᵢ])≈g(Eₚ[yᵢ]) and G(Var[yᵢ])≈G(Varₚ[yᵢ]) by construction. Therefore, prior knowledge about the coefficients in the GLMs can be used to improve the estimation of the treatment effect. However, machine learning models may not generalize perfectly to data outside of the training set. Typically, the generalization performance of a model is measured by holding out some data from the model training phase so that the held-out data can be used to test the performance of the model. For example, suppose that there are one or more control arms from historical clinical trials in addition to the generative model. Then, the c coefficients in accordance with various embodiments of the invention can be estimated by fitting a reduced GLM on the historical control arm data:

$$g ( E [ {y}_{i} ] ) = a + ( {c}_{0} + {\sum }_{j} {c}_{j} {x}_{0 , ij} ) g ( {E}_{p} [ {y}_{i} ] ) , \qquad ( xiv )$$

for the mean or:

$$G ( Var [ {y}_{i} ] ) = α + ( {γ}_{0} + {\sum }_{j} {γ}_{j} {x}_{0 , ij} ) G ( {Var}_{p} [ {y}_{i} ] ) , \qquad ( xv )$$

for the variance. This is particularly useful in a Bayesian framework, in which a distribution π(a, c) or π(a, γ) can be estimated for these coefficients using the historical data, where the data-driven prior distribution can be used in a Bayesian analysis of the RCT. Essentially, this uses the historical data to determine how well the generative model is likely to generalize to new populations, and then applies this information to the analysis of the RCT. In the limit that π(a, c)→δ(a−0)δ(c−1), then digital twins in accordance with a variety of embodiments of the invention can become substitutable for actual control subjects in the RCT. As a result, the better the generative model, the fewer control subjects required in the RCT. In some embodiments, similar approaches could be used to include prior information on any coefficients that are active when wᵢ=0, including the d coefficients.

Examples of workflows for frequentist and Bayesian analyses of clinical trials that incorporate digital twins to estimate treatment effects in accordance with various embodiments of the invention are described below. For a frequentist case for a continuous endpoint, consider a simple example:

$$E [ {y}_{i} ] = a + {b}_{0} {w}_{i} + {c}_{0} {E}_{p} [ {y}_{i} ] \qquad ( xvi )$$

$$while :$$

$$Var [ {y}_{i} ] = {σ}^{2} \qquad ( xvii )$$

assuming no interactions and homoskedastic errors. One skilled in the art will recognize how this can be applied to the more general case captured by Equation xii and Equation xiii. In numerous embodiments, simple analyses can lead to results that are more easily interpreted. This model implies a normal likelihood,

$${y}_{i} ∼ N ( a + {b}_{0} {w}_{i} + {c}_{0} {E}_{p} [ {y}_{i} ] , {σ}^{2} ) \qquad ( xviii )$$

such that the model can be fit (e.g., by maximum likelihood). There are two situations to consider: (1) the design of the trial has already been determined by some method prior to incorporating the digital twins such that the digital twins can be used to increase the statistical power of the trial, and/or (2) the trial needs to be designed so that it incorporates digital twins to achieve an efficient design with sufficient power. In the case of a continuous endpoint, the statistical power of the trial will depend on the correlation between yᵢ and Eₚ[yᵢ], which can be estimated from historical data, and is a function of the magnitude of the treatment effect. In a variety of embodiments, analytical formulas can be derived in this special case. Alternatively, or conjunctively, computer simulations can be utilized in the general case.

Once the trial is designed, patients are enrolled and followed until their outcome is measured. In some cases, patients may not be able to finish the trial and various methods (such as Last Observation Carried Forward) need to be applied in order to impute outcomes for the patients who have not finished the trial, as in most clinical trials. In a number of embodiments, GLMs can be fit to the data from the trial to obtain point estimates \hat{b}₀ and uncertainties δ_{b}_{₀} for the treatment effect. The ratio \hat{b}₀/δ_{b}_{₀} follows a Student's t-distribution which can be used to compute a p-value p_{b}_{₀} and the null-hypothesis that there is no treatment effect can be rejected if p_{b}_{₀}≤ [symbol] in which [symbol] is the desired control of the type-I error rate. This approach is guaranteed to control the type-I error rate, whereas the realized power will be related to the out-of-sample correlation of yᵢ and Eₚ[yᵢ] and the true effect size.

In the Bayesian case for a continuous endpoint with homoskedastic errors, assume a simple analysis,

$$E [ {y}_{i} ] = a + {b}_{0} {w}_{i} + {c}_{0} {E}_{p} [ {y}_{i} ] \qquad ( xix )$$

$$Var [ {y}_{i} ] = {σ}^{2} . \qquad ( xx )$$

In certain embodiments, the simple analysis can lead to results that are more easily interpreted. This model implies a normal likelihood,

$${y}_{i} ∼ N ( a + {b}_{0} {w}_{i} + {c}_{0} {E}_{p} [ {y}_{i} ] , {σ}^{2} ) \qquad ( xxi )$$

but processes in accordance with various embodiments of the invention can use a Bayesian approach to fit it instead of the method of maximum likelihood. In particular, with historical data representing the condition wᵢ=0 that was not used to train the generative model, processes in accordance with many embodiments of the invention can fit the model,

$$E [ {y}_{i} ] = a + {c}_{0} {E}_{p} [ {y}_{i} ] \qquad ( xxii )$$

to the historical data in order to derive prior distributions for the analysis of the RCT. To do so, pick a prior distribution π₀(a, c₀, σ²) such as (but not limited to) a Normal-Inverse-Gamma prior or another appropriate prior distribution. As there are no data to inform the parameters of the prior before analyzing the historical data, processes in accordance with several embodiments of the invention can use a diffuse or default prior. In numerous embodiments, Bayesian updates to the prior distribution can be computed from the historical data to derive a new distribution π_{H}(a, c₀, σ²), in which the subscript H can be used to denote that this distribution was obtained from historical data. Processes in accordance with numerous embodiments of the invention can then specify prior distributions π₀(b₀) for the treatment effects. This could also be derived from data in accordance with many embodiments of the invention if it's available, or a diffuse or default prior could be used. The full prior distribution is now π_{H}(a, c₀, σ²)π₀(b₀). In various embodiments, such distributions can be used to compute the expected sample size in order to design the trial, as in typical Bayesian trial designs. Once the trial is designed, patients can be enrolled and followed until their outcome is measured. In some cases, patients may not be able to finish the trial and various methods (such as Last Observation Carried Forward) can be applied in order to impute outcomes for the patients who have not finished the trial, as in most clinical trials.

In numerous embodiments, GLMs can be fit to obtain posterior distributions π_{RCT}(a, b₀, c₀, σ²) for the parameters. As in a typical Bayesian analysis, the treatment can be declared effective if Prob(b₀≥0) exceeds a pre-specified threshold in accordance with a number of embodiments of the invention.

There are advantages and disadvantages to the frequentist and Bayesian methods that are captured through these simple examples. The frequentist approach to including digital twins in the analysis of an RCT leads to an increase in statistical power while controlling the type-I error rate. If desired, it's also possible to use the theoretical increase in statistical power to decrease the number of subjects required for the concurrent control arm, although this cannot be reduced to zero concurrent control subjects. The Bayesian approach borrows more information about the generalizability of the model used to create the digital twins (e.g., from an analysis of historical data) and, as a result, can increase the power much more than the frequentist approach. In addition, the use of Bayesian methods in accordance with numerous embodiments of the invention can enable one to decrease the size of the concurrent control arm even further. However, the increase in power/decrease in required sample size can come at the cost of an uncontrolled type-I error rate. Therefore, processes in accordance with many embodiments of the invention can perform computer simulations of the Bayesian analysis to estimate the type-I error rate so that the operating characteristics of the trial can be described.

As a final example, it is helpful to consider a simple case in which a GLM is also used for the variance. For example, consider the models

$$E [ {y}_{i} ] = a + {b}_{0} {w}_{i} + {c}_{0} {E}_{p} [ {y}_{i} ] \qquad ( xxiii )$$

$$and$$

$$log Var [ {y}_{i} ] = α + {β}_{0} {w}_{i} + {γ}_{0} {log}_{p} Var [ {y}_{i} ] , \qquad ( xxiv )$$

which reflect the likelihood:

$${y}_{i} ∼ N ( a + {b}_{0} {w}_{i} + {c}_{0} {E}_{p} [ {y}_{i} ] , {e}^{α + {β}_{0} {w}_{i} + {γ}_{0} log {Var}_{p} [ {y}_{i} ]} ) . \qquad ( xxv )$$

Models in accordance with a number of embodiments of the invention can allow for heteroskedasticity in which the variance of the outcome is correlated with the variance predicted by the digital twin model, and in which the variance may be affected by the treatment. In several embodiments, a system of GLMs can be fit (e.g., using maximum likelihood, Bayesian approaches, etc.), as was the case for the simpler model. One skilled in the art will clearly recognize that one could also include the interaction or other terms in order to model more complex relationships if necessary. In addition, one skilled in the art will also recognize that including interactions can lead to estimates of conditional average treatment effects in addition to average treatment effects.

An example of borrowing information from digital twins to estimate treatment effects in accordance with an embodiment of the invention is illustrated in FIG. 3B. In the first part 315, a generative model of the control condition is trained using historical data from previously completed clinical trials, electronic health records, or other studies. In the second part 320, if the analysis to be performed is Bayesian, predictions from the generative model are compared to historical data that were not used to train the model in order to obtain a prior distribution capturing how well the predictions generalize to new populations. A frequentist analysis does not need to obtain a prior distribution. In the third part 325, a randomized controlled trial is conducted (potentially with unequal randomization), digital twins are generated for each subject in the trial, and all of the data are incorporated into a statistical analysis (including the prior from part 320 if the analysis is Bayesian) to estimate the treatment effects. Bayesian methods, analytical calculations, or the bootstrap may be used to estimate uncertainties in the treatment effects, and decision rules based on p-values or posterior probabilities may be applied.

Generative models implemented in accordance with many embodiments of the invention are not limited to use within RCTs. Accordingly, it should be appreciated that applications described herein may also be implemented outside the context of clinical trials. Moreover, while specific generative model configurations are described above in FIGS. 1-3B, any of a variety of model configurations can be utilized to estimate treatment effects as appropriate to the requirements of specific applications.

### II. Prognostic Covariate Adjustment (PROCOVA)

Covariate adjustments for RCTs implemented in accordance with multiple embodiments of the invention may effectively refer to transformations of covariates. In particular, transformations that can yield significant variance reduction for the treatment effect estimator may include but are not limited to expected control outcomes conditional on the covariates. More formally, systems may use prognostic scores as covariates in mean models. The application of generative models (including but not limited to optimized AI algorithms trained on historical control data) to define transformations can enable systems configured in accordance with multiple embodiments of the invention to obtain valid, efficient, and powerful treatment effect inferences via singular adjustments.

In accordance with many embodiments of the invention, PROCOVA methods may be configured to use historical data and prognostic modelling to decrease the uncertainty associated with treatment effect estimates. Systems and methods in accordance with certain embodiments of the invention, when following PROCOVA processes, may implement least squares regression analysis of RCTs. Additionally or alternatively, PROCOVA processes may use heteroskedasticity-consistent (HC) standard errors to quantify the uncertainty associated with treatment effect estimators. Heteroskedastic variables may specifically be derived in response to non-constant variance among participants to RCTs.

### A. Generalized PROCOVA

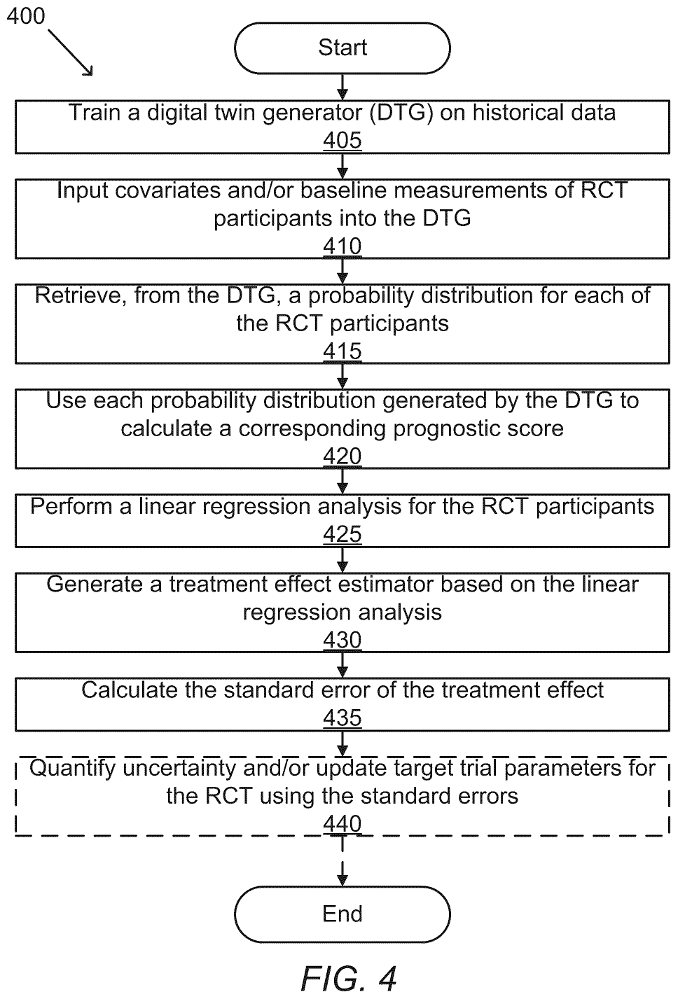

An example of the PROCOVA process applied, in accordance with multiple embodiments of the invention, to summarizing linear regression analysis of RCTs, is illustrated in FIG. 4. Process 400 trains (405) a digital twin generator (DTG) on historical control data. In accordance with many embodiments of the invention, historical control data may include but is not limited to control arm data from historical control arms of previous RCT trials. In some embodiments of the invention, training (405) DTGs may not involve any RCT (e.g., treatment) data. In doing so, the DTGs may be completely prespecified. Process 400 inputs (410) covariates and/or baseline measurements of RCT participants into the DTG. In accordance with some embodiments of the invention, the sole inputs for trained DTGs may be participant covariates and baseline measurements xᵢ. As such, they may avoid introducing any bias in treatment effect inferences for the RCT.

Process 400 retrieves (415), from the DTG, a probability distribution for each of the RCT participants. In accordance with many embodiments, the probability distributions may represent the potential outcomes for the RCT participants, if they were assigned to the control group of an RCT. Probability distributions generated in accordance with certain embodiments of the invention may consider each participant's fixed timepoint post-treatment assignment. Process 400 may represent the probability distributions by the cumulative distribution function (CDF) F_{i,0}: [symbol] →(0,1).

Process 400 uses (420) each probability distribution (i.e., CDF, F_{i,0}) generated by the DTG to calculate a corresponding prognostic score mᵢ=∫_{-∞}^{∞}rdF_{i,0}(r) for the associated RCT participant. In accordance with various embodiments of the invention, prognostic scores may be calculated via Monte Carlo samples (i.e., by obtaining independent samples from each distribution F_{i,0} and calculating the averages of the respective samples). In doing so, process 400 may effectively summarize a large number of covariates into a scalar that is correlated with the observed outcomes. Additionally or alternatively, the prognostic scores may be calculated for all RCT participants independently of the arm of the RCT they end up assigned to (i.e., μᵢ being independent of wᵢ).

Process 400 performs (425) a linear regression analysis for the RCT participants based on the prognostic scores. In accordance with numerous embodiments, regression analysis may be represented in matrix form. For example, there may be N participants in a prospective RCT such that a first vector is an N×1 vector, representing the treatment effect (i.e., outcome to be extrapolated by the regression analysis) for each participant. In many embodiments of the invention, the linear regression analysis for each participant i may be based on vectors of predictors vᵢ. In addition to the prognostic scores (mᵢ), the vector of predictors for participants may include but is not limited to treatment indicators (wᵢ). Performing (425) linear analyses in accordance with various embodiments of the invention may include but is not limited to performing weighted linear analyses (i.e., Weighted PROCOVA) as described below. RCTs configured in accordance with many embodiments of the invention may apply treatments (i.e., wᵢ) randomly. Additionally or alternatively, the treatments may be applied in a manner independent to both pre-treatment covariates (i.e., Xᵢ) and the potential outcomes. In accordance with certain embodiments, potential outcomes may include but are not limited to yᵢ⁽⁰⁾, which can represent the potential outcome(s) when participant i is assigned to a control group, and yᵢ⁽¹⁾, when participant i is assigned to a treatment group.

Process 400 generates (430) a treatment effect estimator (\hat{β}₁) based on the linear regression analysis. As indicated above, treatment effect estimators (or PROCOVA) in accordance with many embodiments of the invention presume a working model Yᵢ=β₀+β₁wᵢ+β₂μᵢ+ϵᵢ where Yᵢ, wᵢ, and μᵢ are a subject's outcome, treatment status, and prognostic score, respectively and ϵᵢ is a noise term. This model can be fit via ordinary least-squares and the value of β₁ can be taken as the point estimate of the treatment effect, \hat{β}₁. In several embodiments, the linear regression analyses may produce fitted regression models used for producing the treatment effect estimator(s). This impact of the treatment effect estimators is expounded upon in Schuler, A. et al. (2021) “Increasing the efficiency of randomized trial estimates via Linear Adjustment for a prognostic score,” The International Journal of Biostatistics, 18(2), pp. 329-356. Available at: https://doi.org/10.1515/ijb-2021-0072, incorporated by reference in its entirety.

Process 400 calculates (435) the standard error of the treatment effect. The standard error of treatment effects calculated in accordance with a number of embodiments of the invention may take on different forms including but not limited to heteroskedasticity-consistent (HC) standard errors (e.g., HC0, HC1, HC2, HC3). Additional prospective steps for computing HC standard errors are disclosed in Romano, J. P. and Wolf, M. (2017) “Resurrecting weighted least squares,” Journal of Econometrics, 197(1), pp. 1-19, Available at: https://doi.org/10.1016/j.jeconom.2016.10.003, incorporated by reference in its entirety.

In accordance with many embodiments of the invention, HC errors may be calculated to follow regulatory guidance standards regarding covariate adjustments for RCTs, including but not limited to those determined by the European Medicines Agency and the Food and Drug Administration (e.g., European Medicines Agency (2015). Guideline on Adjustment for Baseline Covariates in Clinical Trials; Food and Drug Administration, US Department of Health and Human Services, Center for Drug Evaluation and Research (CDER), and Center for Biologics Evaluation and Research (CBER) (2023). Adjusting for Covariates in Randomized Clinical Trials for Drugs and Biological Products: Guidance for Industry) incorporated by reference in their entireties.

In accordance with certain embodiments of the invention, the calculated (435) standard error may be used for various applications. Process 400 may quantify (440) uncertainty for with the RCT using the standard errors. Additionally or alternatively, process 400 may update target trial parameters for the RCT according to any quantified uncertainty. In accordance with many embodiments of the invention, prognostic model estimates for treatment effects and estimates for their uncertainty can be used to perform hypothesis tests in order to create decision rules that guide target trials.

While specific processes for determining treatment effects are described above in FIG. 4, any of a variety of processes can be utilized to implement randomized controlled trials as appropriate to the requirements of specific applications. In certain embodiments, steps may be executed or performed in any order or sequence not limited to the order and sequence shown and described. In a number of embodiments, some of the above steps may be executed or performed substantially simultaneously where appropriate or in parallel to reduce latency and processing times. In some embodiments, one or more of the above steps may be omitted.

Defined in terms of the distributions of covariates and potential outcomes, covariate adjustment with prognostic scores can consistently yield smaller mean squared errors compared to the other methods, across multiple different settings. These observations demonstrate theoretical results that PROCOVA can minimize asymptotic variance among a class of estimators, the uncertainty in the treatment effect estimator may be minimized when the prognostic model predicts the participants' control potential outcomes, and/or gains can be realized in efficiency even with imperfect prognostic models/in the presence of heterogeneous effects. The observations also establish that PROCOVA may decrease the variance of the treatment effect estimators proportional to the squared correlation of the prognostic score with the outcome. In doing so, PROCOVA may guarantee unbiasedness, control of Type-I error rates, and/or desirable confidence interval coverage.

PROCOVA can unite AI and historical control data to decrease uncertainty in treatment effect inferences for RCTs. For example, DTGs implemented in accordance with certain embodiments of the invention may be implemented by a variety of mathematical and/or computational means. Additionally or alternatively, a priori variance reduction and/or power boosts may cast in terms of goodness-of-fit metrics for DTGs on validation data. The benefits of adjusting via the prognostic score(s) can increase as functions of the correlation(s) between the prognostic score(s) and the outcomes. Moreover, by adjusting based solely on the prognostic score(s) (instead of the entire predictor vector vᵢ), systems operating in accordance with numerous embodiments of the invention can use fewer degrees of freedom, which in turn can increase power (e.g., when M is large). PROCOVA implemented in accordance with many embodiments of the invention can be semiparametric efficient when the treatment effects are constant, the historical data follows the same distributions as the trial control arms, and/or the prognostic models improve with the amount of historical data. This implies that the power of a trial using PROCOVA will be greater than or equal to the power of any other trial design that controls the Type-I error rate.

### B. Weighted PROCOVA

Weighted PROCOVA may refer to a type of weighted linear regression for modelling the relationships between actual (treated and control) outcomes and vectors of predictor variables defined (in part) based on summaries of the digital twins of trial participants (e.g, prognostic scores and the variances of participants' digital twins). outcomes (i.e., prognostic scores) and vectors (vᵢ) of predictor variables, also referred to as “predictors” and/or “predictor variables” in this disclosure. Examples of predictors for outcomes may include but are not limited to means (pi) and treatment indicators (wᵢ). Various implementations of Weighted PROCOVA may be implemented in accordance with certain embodiments of the invention, such as those disclosed in U.S. patent application Ser. No. 18/330,259, entitled “Systems and Methods for Adjusting Randomized Experiment Parameters for Prognostic Models,” filed Jun. 6, 2023; U.S. patent application Ser. No. 18/308,619, entitled “Systems and Methods for Adjusting Randomized Experiment Parameters for Prognostic Models,” filed Apr. 27, 2023, both of which are incorporated by reference in their entirety for all purposes.

As suggested above, under linear regression methods, the μ₀(xᵢ) (the expected outcome for participant i when sorted into the control group) and/or μ₁(xᵢ) (the expected outcome for participant i when sorted into the treatment group) can be modeled (via predictor vectors vᵢ ∈ [symbol] ^{M+1}, where M represents the number of redictor variables) as linear functions of unknown regression coefficients β=(β₀, . . . , β_{M}) ∈ [symbol] ^{M+1}. As such, systems in accordance with many embodiments of the invention may find optimal (unbiased) treatment effect estimators through finding regression coefficients that minimize loss. Weighted PROCOVA processes may build on weighted linear regression by finding the coefficients β that minimize weighted least squares loss functions.

When the variance of the potential outcomes (conditional on the predictors) is known for the RCT participants, Weighted PROCOVA processes may use the following formula the estimators that minimize weighted least squares loss functions in the target RCT:

$$L ( β ) = {\sum }_{i = 1}^{N} {( {y}_{i} - {v}_{i}^{T} β )}^{2} / {σ}_{i}^{2} = {( y - V β )}^{T} {Ω}^{- 1} ( y - V β ) , \\ V = ( \begin{matrix}{v}_{1}^{T} \\ ⋮ \\ {v}_{N}^{T}\end{matrix} ) , y = {( {y}_{i} , … , {y}_{N} )}^{T} , Ω = ( \begin{matrix}{σ}_{1}^{2} & … & 0 \\ ⋮ & ⋱ & ⋮ \\ 0 & … & {σ}_{N}^{2}\end{matrix} ) \qquad ( 1 )$$

where N denotes the total number of participants in the RCT, y denotes the observed outcomes, V denotes the predictor variables; Ω denotes a diagonal matrix with the variances in outcome for each participant making up the diameter, and β denotes the vector of regression coefficients for the expected outcomes.

Consideration of inferences for β in the case of known σᵢ² may be used to configure “plug-in” inferential strategies in the case of unknown σᵢ². Specifically, systems implemented in accordance with several embodiments of the invention may obtain initial estimates ( [symbol] ) for σᵢ² based on samples retrieved RCT data. Using these estimates in the above loss function, systems may derive the estimator \hat{β} that minimizes the loss function, taking the form:

$$\hat{β} = {( {V}^{T} {\hat{Ω}}^{{}_{- 1}} V )}^{- 1} {V}^{T} {\hat{Ω}}^{{}_{- 1}} y , \\ \hat{Ω} = ( \begin{matrix} & … & 0 \\ ⋮ & ⋱ & ⋮ \\ 0 & … & \end{matrix} ) \qquad ( 2 )$$

To obtain values for variance estimators [symbol] for weighted linear regression, systems in accordance with multiple embodiments of the invention may model transformations of σᵢ² as functions of covariates (skedastic function models). Systems implemented in accordance with many embodiments of the invention may use logarithmic transformations for skedastic function models, where the models have the form log(σᵢ²)=uᵢ^{T}γ where: uᵢ^{T}∈R^{L+1} denotes the predictor vector for the skedastic function model; the first entry in each uᵢ is equal to 1; and γ∈R^{L+1} is a vector of unknown coefficients. Therefore, under the logarithmic transformation, and using estimator \hat{γ} for γ, the estimator for σᵢ² may take the form [symbol] =exp(uᵢ^{T}\hat{γ}).

In accordance with many embodiments of the invention, the loss function for both β and γ may take the form:

$$L ( β , γ ) = {\sum }_{i = 1}^{N} {[ log \{ {f}_{i} ( β , γ ) \} ]}^{2} + {\sum }_{i = 1}^{N} {f}_{i} ( β , γ ) , {f}_{i} ( β , γ ) = exp ( - {u}_{i}^{T} γ ) {( {y}_{i} - {v}_{i}^{T} β )}^{2} . \qquad ( 3 )$$

Using this formula, in accordance with many embodiments of the invention, minimizing the first term of the formula (Σᵢ₌₁^{N} [log{ƒᵢ(β,γ*)}]²) may be considered a linear regression problem for β, using estimate γ* for γ. Additionally or alternatively, the second term of the formula Σᵢ₌₁^{N} ƒᵢ(β*,γ) may be considered a linear regression problem for γ, using estimate β* for β. Systems configured in accordance with various embodiments of the invention may thereby optimize the above formula by iterating between the two regression problems given by these two terms. Therefore, when a minimizing value \hat{γ} is obtained for the second term, the value for [symbol] (i.e., exp(uᵢ^{T}\hat{γ})) may be used to approximate the entry (\hat{Ω}ᵢᵢ) for participant i in the Ω matrix of the weighted least squares loss function of Equation (1).

In accordance with some embodiments of the invention, logarithms of the variances of the digital twins for participants may be used to determine covariates in participant-level variance models for weighted linear regression. In such cases, the prognostic scores may remain the primary covariate adjustments for the mean (i.e., prognostic) models. The inverse of the variance of the probability distributions from DTGs implemented in accordance with many embodiments of the invention may be described, in this disclosure, as a participant's “personalized precision” of a participant's outcome conditional on all their covariates. In addition or alternative to the use of the variances of the digital twins, in some embodiments of the invention, the logarithmic approach may enable logarithms of the personalized precisions to be the covariate(s) for the participant-level variance (i.e., skedastic) models. Additionally or alternatively, (the inverse of) the predictions from the fitted participant-level variance models may be used to define the weights for the weighted regression analyses of RCT data. Nevertheless, this disclosure may primarily describe the use of logarithms of the variances of the digital twins in determining covariates.

In accordance with various embodiments of the invention, Weighted PROCOVA may incorporate additional features, uniquely derived from DTGs, that (along with prognostic scores) address heteroskedasticity and provide more efficient and powerful analysis in RCTs. For each participant i=1, . . . , N at (at least one) fixed timepoint post-treatment, systems configured in accordance with many embodiments of the invention may obtain values for (personalized precision from the inverse of the) inferred variance (sᵢ²=∫_{-∞}^{∞}(r−mᵢ)²dF_{i,0}(r)). In doing so, systems may utilize log(sᵢ²) as the (e.g., sole) predictor for skedastic function models, where for each participant, the predictor vector for the skedastic function models uᵢ=(1, log(sᵢ²))^{T}. In accordance with multiple embodiments of the invention, sᵢ² may be determined via Monte Carlo sampling by obtaining independent samples from each distribution F_{i,0} and calculating the variances of the respective samples.

Given the point estimates \hat{γ}=(U^{T}U)⁻¹U^{T} log{(y−Yy)²} as defined above for uᵢ=(1, log(sᵢ²))^{T}, systems in accordance with some embodiments of the invention may estimate \hat{σ}ᵢ²=e^{\hat{γ}}^{₀}(sᵢ²)^{\hat{γ}}^{₁} and construct \hat{Ω} accordingly to infer the treatment effects in terms of β₁. In order to satisfy regulatory guidelines, inferences on β₁ may be based on the standard error of \hat{β}₁ as calculated using the combination of the HC1 method with \hat{Ω}. The prognostic score mᵢ remains as a covariate in vᵢ.

In accordance with many embodiments of the invention, Weighted PROCOVA may be used to refine calculated estimates including but not limited to expected outcome, conditional on both sets of predictors ( [symbol] (yᵢ|U, V)); and variance, conditional on both sets of predictors (Var(yᵢ| U, V)). Thus, systems in accordance with some embodiments may use two features, generated based on two sets of predictors, in the calculation of the two conditional moments.

Weighted PROCOVA may follow regulatory guidance on covariate adjustment by leveraging AI algorithm(s) trained on historical, out-of-sample control data to summarize the high-dimensional covariate vectors for heteroskedasticity into the inferred variance scalar (sᵢ²) and/or personalized precision scalar (sᵢ⁻²). As will be described elsewhere in this disclosure, weighted regression, as performed in accordance with many embodiments of the invention, does not attempt to adjust for multiple covariates in the participant-level variance model, but instead adjusts solely for a transformation of the (e.g., inferred variance) scalar(s). The participant-level variance models in RCTs may then be specified and/or fitted in a stable and interpretable manner solely as functions of the scalar(s). This serves the purpose of preventing the complications associated with adjusting for multiple covariances in the variance model. This further yields an interpretable weighting mechanism for RCTs, with the strength of the scalar(s) in explaining heteroskedasticity governing the efficiency and power of treatment effect inferences from the weighted regression models. Additionally or alternatively, systems in accordance with many embodiments relate the quality of the DTG predictions to the gain in statistical efficiency (i.e., reduced variance). Finally, Weighted PROCOVA enables one to adapt the above values obtained from DTGs to RCT datasets. This prevents incongruities in the weights, such as assigning less weight to participants whose outcomes are poorly predicted.

The estimated variance \hat{σ}ᵢ² for each participant may be proportional to a power of their sᵢ² values as derived from the DTGs. When DTGs are well-calibrated, systems configured in accordance with some embodiments of the invention can reliably expect \hat{γ}₀≈0 and \hat{γ}₁≈1. Therefore, systems can effectively specify the (i, i) entry of \hat{Ω} as sᵢ². When DTGs are not well-calibrated, systems, by implementing Weighted PROCOVA, may re-calibrate the DTGs based on the variability exhibited in the squared residuals of a RCT, preventing participants with large residuals (with the residual for participant i defined as eᵢ=yᵢ−\hat{γ}ᵢ) from being given large weight (e.g., in cases where the sᵢ² is small).

In many embodiments of the invention, diagnostics can be performed to help recognize potentially undesirable statistical regimes for the application of Weighted PROCOVA. A first set of diagnostics may involve the use of plots and/or statistical tests on residuals from PROCOVA to evaluate evidence for attributes including but not limited to heteroskedasticity. Nevertheless, in accordance with certain embodiments of the invention, Weighted PROCOVA can still be valid under homoskedasticity; however, in such cases, it may be preferable to directly set γ₁=0. A second set of diagnostics may include examination of the variance of the sᵢ² across participants. Specifically, when the variance of the sᵢ² is small then this feature may fail to provide sufficient information for Weighted PROCOVA to improve the quality of treatment effect inferences. A third set of diagnostics may include examination of joint distributions of the inferred weights, inferred variances, personalized precisions, and/or residuals to confirm that large weights are attached to participants for whom the model is more confident with respect to predicting their outcomes.

In accordance with many embodiments of the invention, the relationship between inferred weights and values such as residuals may impact the effectiveness of Weighted PROCOVA. For example, Weighted PROCOVA may be especially effective when participants with small residuals have large inferred weights. Additionally or alternatively, the quality of inferences may be expected to decrease when participants with large residuals have large inferred weights. Finally, it is important to diagnose whether γ₁ is inferred to be negative. In such cases, participants with large digital twin variances may be given more weight even when their squared residuals are also large. This may be undesirable since more weight would ideally be allocated to participants with low variances and small squared residuals.

Weighted PROCOVA may especially benefit from the incorporation of generative AI. Generative AI can effectively summarize the relationships between the variation in the outcomes and all the covariates to construct the scalar feature sᵢ² that is optimized to explain the variation. This advantageous feature can free systems implemented in accordance with some embodiments from having to perform additional calculations, including but not limited to analyzing all the entries in high-dimensional covariate vectors and/or considering large number of coefficients when specifying skedastic function models. This is desirable from a regulatory perspective, as guidance from regulatory agencies indicate that the number of covariates used in the statistical analysis of RCTs should be kept as small as possible. Furthermore, it facilitates the interpretability of the DTG and methodology, especially for non-statisticians. Systems and methods in accordance with multiple embodiments of the invention can gain useful insights in a straightforward manner by comparing the eᵢ² and sᵢ², and considering the inferences on γ₀ and γ₁.

### C. Fixed Function Weighted PROCOVA

In accordance with numerous embodiments of the invention, Fitted Function Weighted PROCOVA methods may be configured to use RCT data and prognostic modelling to decrease the uncertainty in treatment effect estimates. In accordance with many embodiments of the invention, the RCT data used in Fitted Function Weighted PROCOVA methods (also referred to as “target trial data” in this section) may be derived directly from RCTs. Systems and methods in accordance with various embodiments of the invention, when following PROCOVA processes, may involve ordinary least squares regression analysis of the RCTs. Systems may thereby yield, asymptotically, unbiased estimators for treatment effects, control Type I error rates, and/or maintain confidence interval coverage.

In accordance with some embodiments of the invention, Fitted Function PROCOVA processes may follow many of the restrictions described under General and Fixed Function Weighted PROCOVA methods. For example, these processes may use HC standard errors to quantify the uncertainty associated with treatment effect estimators. Further, as indicated above, prognostic models may correspond, but are not limited, to AI-generated digital twin implementations. Examples of statistics (i.e., predictors) for digital twins may include but are not limited to means (μᵢ), standard deviations (σᵢ²), and quantiles of the distribution. In accordance with various embodiments of the invention, Fitted Function Weighted PROCOVA can be viewed as Zero Trust and/or Limited Trust AI solutions.

In accordance with a number of embodiments of the invention, Fitted Function Weighted PROCOVA processes may diverge from Fixed Function Weighted PROCOVA processes in relation to the use of the skedastic functions used. For example, one skedastic function model may also take the form:

$$g ( {σ}_{i}^{2} ) = {γ}_{0} + {γ}_{1} {σ}_{i}^{2} . \qquad ( 4 )$$

Nevertheless, in accordance with several embodiments of the invention, and in contrast to Fixed Function Weighted PROCOVA processes, parameters γ₀ and γ₁ may be inferred in manners dependent on the data for the target trial.

Systems configured in accordance with some embodiments may obtain samples of Monte Carlo (MC) draws from digital twins and/or utilize summaries of MC draws to implement Fitted Function Weighted PROCOVA processes. Additionally or alternatively, Fitted Function Weighted PROCOVA processes may utilize weighted least squares estimators obtained by finding the coefficients that minimize the weighted least squares loss function disclosed above in the target RCT:

$$L ( β ) = {\sum }_{i = 1}^{N} {{C}_{i} ( {Y}_{i} - {X}_{i}^{T} β )}^{2} . \qquad ( 5 )$$

### 1. Fitted Function Implementation

In accordance with various embodiments, identifying the coefficients that minimize the weighted least squares loss function may be based on fixed weights Cᵢ=1/g (σᵢ²). Additionally or alternatively, Cᵢ may be limited to positive quantities and used to derive HC standard errors. Additionally or alternatively, weight coefficients (Cᵢ) may be derived from processes including but not limited to predicted tail-area probabilities or other metrics of uncertainty in the model's predictions.

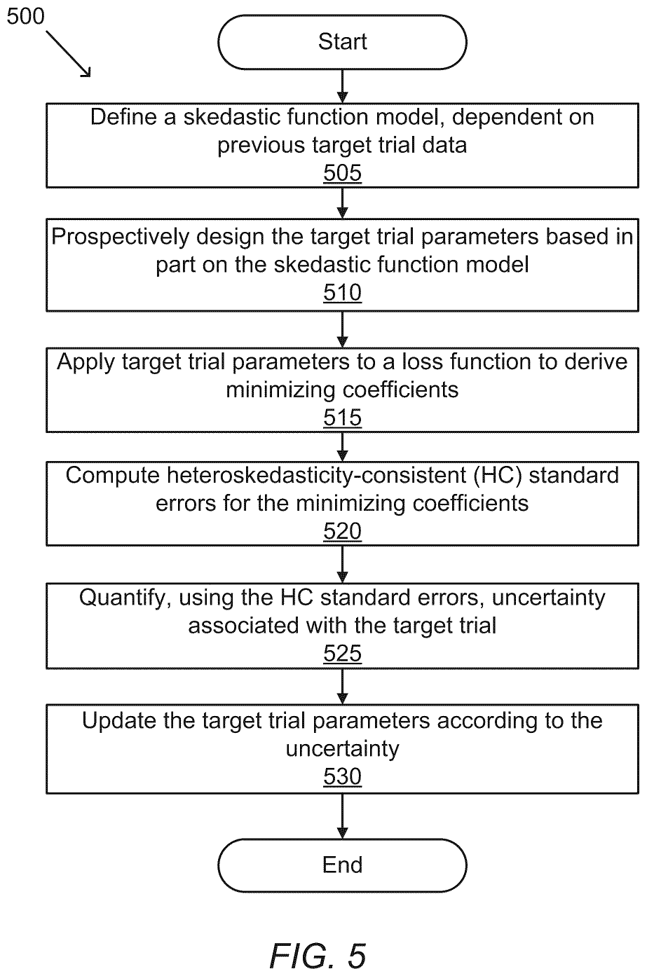

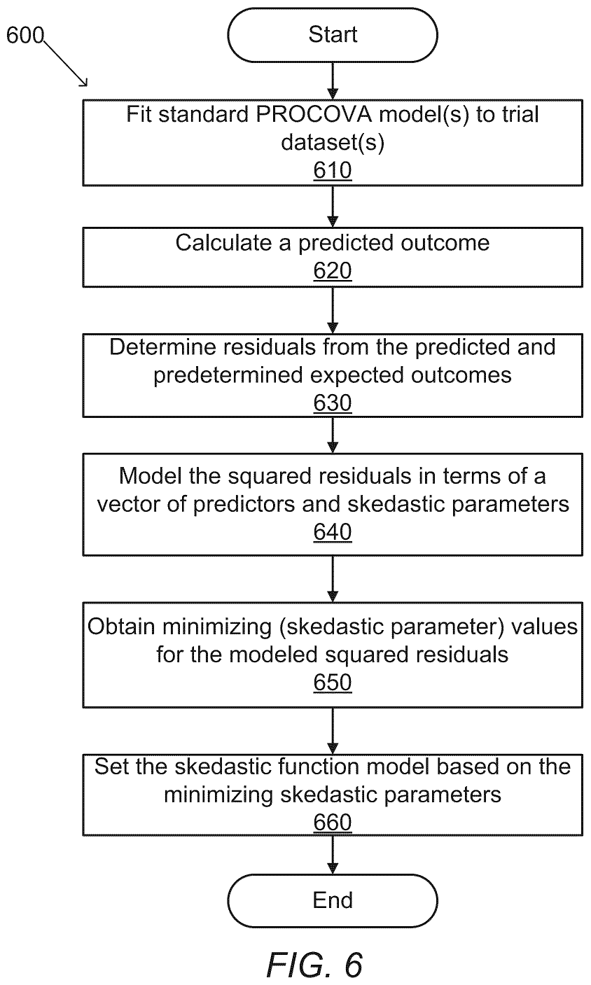

A process for Fitted Function Weighted Prognostic Covariate Adjustments, in accordance with a number of embodiments of the invention, is conceptually illustrated in FIG. 5. Process 500 defines (505) a skedastic function model, dependent on target trial data. Such target trials may include but are not limited to randomized controlled trials (RCTs). In accordance with many embodiments of the invention, skedastic function models may be defined through determining a particular mathematical form for the skedastic function model. In accordance with some embodiments, the initial form may follow standard PROCOVA models as disclosed above. Process 500 may, additionally or alternatively, define the skedastic function model using the process disclosed in FIG. 6.

Process 500 prospectively designs (510) the target trial parameters based in part on the skedastic function model. In accordance with numerous embodiments of the invention, the design for the target trial may be specified based on mathematical formulae and/or computer code for reduction of the expected variance of the treatment effect estimator. The expected variance reduction may yield values for the reduction of the control arm sample size (and thereby the design) for the target trial. Additionally or alternatively, the expected variance reduction may yield a value for the power (of the test of the treatment effect). In accordance with some embodiments of the invention target trial parameters may include but are not limited to data from and/or for the target trial.

Process 500 applies (515) target trial parameters to a loss function to derive minimizing skedastic model and minimizing regression model coefficients. In accordance with certain embodiments of the invention, values for the expected variance reduction in this step may be obtained in multiple ways:

(A) In accordance with certain embodiments, [symbol] (σᵢ²) may represent the limit of the fitted skedastic function model as N→∞. c

$$Ω = E ( {X}_{i} {X}_{i}^{T} ) , {Ω}_{{}^{{s}^{{}_{2}}}} = E ( {s}_{i}^{2} {X}_{i} {X}_{i}^{T} ) . \qquad ( 6 )$$

Specifically, when the diagonal entry in [symbol] [symbol] [symbol] that corresponds to the treatment indicator in Xᵢ is d1, and the diagonal entry in Ω⁻¹Ωₛ_{²}Ω⁻¹ that corresponds to the treatment indicator in Xᵢ is d2, the estimated variance reduction may take the value d1/d2. As the sᵢ² are unknown, these values can be replaced by estimates obtained from other, out-of-sample datasets including but not limited to datasets from other trials. These estimates may, in turn, be used to estimate what the variance reduction would be in the target trial. Other variance reduction systems are disclosed below.

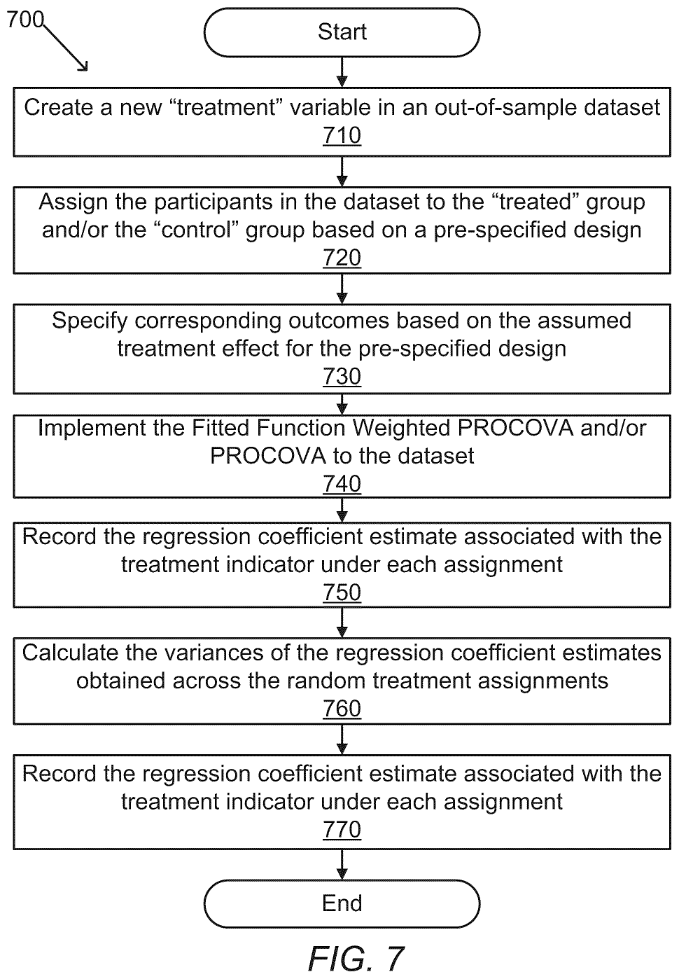

(B) Additionally or alternatively, when the skedastic function model itself is estimated from target trial data, applying (515) target trial parameters to a loss function may, additionally or alternatively, involve the (computational) procedure disclosed in FIG. 7.

Once values for expected variance are obtained, they may be used to derive weights in order to perform the weighted least squares procedure as discussed above. Once the skedastic function model is defined, weights may be set to Cᵢ=1/g (σᵢ²) and used to solve for the values of β that minimize the loss function (i.e., L(β)=Σᵢ₌₁^{N} Cᵢ(Yᵢ−Xᵢ^{T}β)²). Systems and methods configured in accordance with multiple embodiments of the invention may consider additional variants of weights defined based on transformations of the squared residuals. Examples may include but are not limited to logarithmic transformations and square root transformations of the squared residuals. In such cases, models may be fit on the transformed space of the squared residuals. Additionally or alternatively, the models may be back-transformed to obtain transformed models on the original space of the squared residuals. In accordance with many embodiments of the invention, weights Cᵢ may be required to be positive, while transformations may be configured to enforce this constraint. Additionally or alternatively, the transformation that is applied to the squared residuals may also be applied to σᵢ² for model interpretability.

Process 500 computes (520) heteroskedasticity-consistent (HC) standard errors for the minimizing coefficients. Additional prospective steps for computing HC standard errors are disclosed in Romano, J. P. and Wolf, M. (2017) “Resurrecting weighted least squares,” Journal of Econometrics, 197(1), pp. 1-19, Available at: https://doi.org/10.1016/j.jeconom.2016.10.003, incorporated by reference in its entirety. Process 500 quantifies (525), using the standard errors, uncertainty associated with the target trial. Process 500 updates (530) the target trial parameters according to the uncertainty. In accordance with many embodiments of the invention, prognostic model estimates for treatment effects and estimates for their uncertainty can be used to perform hypothesis tests in order to create decision rules that guide target trials.

Skedastic function models configured in accordance with some embodiments of the invention may have no significant relationships between the predictors in the Zᵢ and the squared residual eᵢ². In such cases, all of the entries in γ that correspond to the predictors in the Zᵢ (excluding the intercept term) may effectively be estimated as zero from the historical dataset. The only non-zero coefficient may then be the intercept term γ₀, thereby making the Fitted Function Weighted PROCOVA equivalent to a PROCOVA with a constant variance.

A process for defining skedastic function models, in accordance with multiple embodiments of the invention, is illustrated in FIG. 6. Process 600 fits (610) standard PROCOVA model(s) to trial dataset(s). Process 600 may fit standard PROCOVA models to a trial dataset by finding values of β (regression coefficients) that minimize the sum of squares for the standard PROCOVA models in the following formula:

$${\sum }_{i = 1}^{N} {( {Y}_{i} - {X}_{i}^{T} β )}^{2} . \qquad ( 7 )$$

As such, the estimated value for β may take the form:

$$\hat{β} = {( {X}^{T} X )}^{- 1} {X}^{T} Y \\ where \\ X = ( \begin{matrix}{X}_{1}^{T} \\ {X}_{2}^{T} \\ ⋮ \\ {X}_{N}^{T}\end{matrix} ) , Y = ( {Y}_{1} , … , {Y}_{N} ) . \qquad ( 8 )$$

For each participant i in the trial process 600 calculates (620) their predicted outcome based on the fitted PROCOVA model. In accordance with many embodiments, predicted outcomes may be derived from the formula:

$${\hat{Y}}_{i} = {X}_{i}^{T} \hat{β} . \qquad ( 9 )$$

Process 600 determines (630) residuals from the predicted and observed (Yᵢ) outcomes in the trial dataset:

$${e}_{i} = {Y}_{i} - {\hat{Y}}_{i} . \qquad ( 10 )$$

Process 600 models (640) a transformation of the squared residuals eᵢ² in terms of a vector of predictors (Vᵢ), including predictors of interest, and skedastic function model parameters. In doing so, process 600 obtains (650) minimizing (skedastic function model parameter) values for the modeled transformation of the squared residuals. The minimizing (skedastic function model parameter) values may correspond to the coefficient minimizing vector of parameter values that minimizes the sum of squares for the modelled squared residuals. One example of modelling transformations of the squared residuals may take the form:

$${e}_{i}^{2} = {V}_{i}^{T} γ = {γ}_{0} + {γ}_{1} {σ}_{i}^{2} \qquad ( 11 )$$

where Vᵢ=(1, σᵢ²) and the minimizing skedastic function model parameter, γ=(γ₀, γ₁). The estimate may therefore be the vector of parameter values that minimizes the sum of squares, as in the reorganized:

$${\sum }_{i = 1}^{N} {( {e}_{i}^{2} - {γ}_{0} - {γ}_{1} {σ}_{i}^{2} )}^{2} \qquad ( 12 )$$

wherein the estimate for the minimizing skedastic function model parameter vector would be:

$$\hat{γ} = {( {V}^{T} V )}^{- 1} {V}^{T} E \qquad ( 13 )$$

$$where :$$

$$V = ( \begin{matrix}{V}_{1}^{T} \\ {V}_{2}^{T} \\ ⋮ \\ {V}_{N}^{T}\end{matrix} ) , E = ( {e}_{1}^{2} , … , {e}_{N}^{2} ) .$$

Potential (frequentist) methods of estimating this vector are disclosed in the next section.

Process 600 sets (660) the skedastic function model based on the minimizing skedastic function model parameters. In doing so, the weights of the model may be a function of \hat{γ} (e.g., g(σᵢ²)=\hat{γ}₀+\hat{γ}₁σᵢ²).

A process for obtaining expected variance reductions in accordance with certain embodiments of the invention is illustrated in FIG. 7. Process 700 creates (710) a new “treatment” variable in an out-of-sample dataset. The out-of-sample dataset may be limited to only control subjects. Process 700 repeatedly assigns (720) the participants in the dataset to the “treated” group or the “control” group based on a pre-specified design. Process 700 specifies (730) a corresponding outcome for each participant under each assignment based on the assumed treatment effect. Process 700 implements (740) the Fitted Function Weighted PROCOVA and/or PROCOVA to the dataset. In implementing (740) this, under each assignment, process may include the treatment indicator(s) as predictor(s). Process 700 records (750) the regression coefficient estimate associated with the treatment indicator under each assignment. Process 700 may repeat the implementation (740) and recordation (750) multiple times across random treatment assignments and modify (755) the assignments. Process 700 calculates (760) the variances of the regression coefficient estimates obtained from Fitted Function Weighted PROCOVA and/or PROCOVA, respectively, across the random treatment assignments. Process 700 determines (770) the (expected) variance reduction of Fitted Function Weighted PROCOVA with respect to PROCOVA. Potential methods of determining the variance reduction of Weighted PROCOVA (including but not limited to Fitted Function Weighted PROCOVA) are disclosed below.

### 2. Fitted Function Frequentist Approach

Systems and methods in accordance with many embodiments of the invention may establish the frequentist properties of the skedastic function model coefficient estimators \hat{γ} and/or the regression coefficient estimators \hat{β} under two perspectives. The first is the finite-sample perspective conditional on U and V. The second is the asymptotic perspective, integrating over the distributions of the uᵢ and vᵢ and taking N→∞. For both cases our derivations are performed for the first iteration of Weighted PROCOVA, so as to facilitate the algebra. Under the finite-sample perspective, systems may calculate E(\hat{γ}|U, V), E(\hat{β}| U, V), and Var(\hat{γ}|U, V), while assuming that the linear regression model for observed outcomes (yᵢ=vᵢ^{T}β+ϵᵢ with ϵᵢ˜N(0, σᵢ²) independently), is correctly specified. Under the asymptotic perspective, systems may identify conditions on the predictors and the participant-level variances that are sufficient for the convergence of the expectation of \hat{γ} as will be depicted below. The asymptotic covariance matrix of \hat{β}, used to characterize the variance reduction of Weighted PROCOVA compared to PROCOVA, is further described below and in the next section.

### a) Proposition 1

For the (idempotent and symmetric) “hat-matrix” H=V(V^{T}V)⁻¹V^{T}; hᵢⱼ denotes entry (i, j) of H; each diagonal entry hᵢᵢ of H corresponds to the leverage for participant i; and hᵢᵢ=Zⱼ₌₁^{N} hᵢⱼ²=hᵢᵢ²+Σ_{j≠1}^{N} hᵢⱼ², so that 0≤hᵢᵢ≤1 and all the hᵢⱼ approach 0 as the hᵢᵢ values approach 0 or 1. Accounting for the above, for the estimators \hat{γ}=(U^{T}U)⁻¹U^{T} log{(y−Hy)²}:

$$𝔼 ( {\hat{γ}}_{0} \vert U , V ) = {[ N {\sum }_{k = 1}^{N} {\{ log ( {s}_{k}^{2} ) - \overline{log ( {s}^{2} )} \}}^{2} ]}^{- 1} \times ( [ {\sum }_{k = 1}^{N} {\{ log ( {s}_{k}^{2} ) \}}^{2} ] {\sum }_{i = 1}^{N} log \{ {( 1 - {h}_{ii} )}^{2} {σ}_{i}^{2} {\sum }_{k \neq i} {h}_{i k}^{2} {σ}_{k}^{2} \} - N \overline{log ( {s}^{2} )} {\sum }_{i = 1}^{N} [ log ( {s}_{i}^{2} ) log \{ {( 1 - {h}_{i i} )}^{2} {σ}_{i}^{2} + {\sum }_{k \neq i} {h}_{i k}^{2} {σ}_{k}^{2} \} ] ) - ( {γ}_{EM} + log 2 ) 𝔼 ( {\hat{γ}}_{1} \vert U , V ) = {[ {\sum }_{k = 1}^{N} {\{ log ( {s}_{k}^{2} ) - \overline{log ( {s}^{2} )} \}}^{2} ]}^{- 1} \times ( {\sum }_{i = 1}^{N} [ \{ log ( {s}_{i}^{2} ) - \overline{log ( {s}^{2} )} \} log \{ {( 1 - {h}_{ii} )}^{2} {σ}_{i}^{2} + {\sum }_{k \neq i} {h}_{i k}^{2} {σ}_{k}^{2} \} ] )$$

where γ_{EM}≈0.577 is the Euler-Mascheroni constant.

### b) Proposition 2

Let [(Z₁, . . . , Z_{N})^{T}|U, V] be Multivariate Normal with [symbol] (Zᵢ|U, V)=0, Var(Zᵢ|U, V)=1, and:

$$Cov ( {Z}_{i} , {Z}_{j} \vert U , V ) = \frac{{\sum }_{k \neq i , j} {h}_{i k} {h}_{jk} {σ}_{k}^{2} - {h}_{ij} ( 1 - {h}_{i i} ) {σ}_{i}^{2} - {h}_{ij} ( 1 - {h}_{jj} ) {σ}_{j}^{2}}{\sqrt{{( 1 - {h}_{i i} )}^{2} {σ}_{i}^{2} + {\sum }_{k \neq i} {h}_{i k}^{2} {σ}_{k}^{2}} \sqrt{{( 1 - {h}_{jj} )}^{2} {σ}_{j}^{2} + {\sum }_{k \neq j} {h}_{jk}^{2} {σ}_{k}^{2}}}$$

for distinct i, j ∈ {1, . . . , N}. Then

$$Var ( {\hat{γ}}_{0} \vert U , V ) = \frac{{\overline{\{ log ( {s}^{2} )} \}}^{2} Var [ {\sum }_{i = 1}^{N} \{ 1 - log ( {s}_{i}^{2} ) \} log ( {Z}_{i}^{2} ) \vert U , V ]}{{[ {\sum }_{i = 1}^{N} {\{ log ( {s}_{i}^{2} ) - \overline{log ( {s}^{2} )} \}}^{2} ]}^{2}}$$

$$and$$

$$Var ( {\hat{γ}}_{1} \vert U , V ) = \frac{Var [ {\sum }_{i = 1}^{N} \{ log ( {s}_{i}^{2} ) - \overline{log ( {s}^{2} )} \} log ( {Z}_{i}^{2} ) \vert U , V ]}{{[ {\sum }_{i = 1}^{N} {\{ log ( {s}_{i}^{2} ) - \overline{log ( {s}^{2} )} \}}^{2} ]}^{2}} .$$

### c) Proposition (Theorem) 3

The estimator \hat{β}=(V^{T}Ω⁻¹V)⁻¹V^{T}Ω⁻¹y is unbiased conditional on U and V, i.e., [symbol] (\hat{β}|U, V)=β. A consequence is super-population unbiasedness of \hat{β}.

Corollary 3.1 The estimator \hat{β}=(V^{T}\hat{Ω}⁻¹V)⁻¹V^{T}Ω⁻¹y is unbiased unconditional on U and V, i.e., [symbol] (\hat{β})=β.

### d) Proposition 4

In accordance with many embodiments of the invention, asymptotic expectations for \hat{γ} can be established under the following assumptions on the predictors U and V, and the participant-level variances σᵢ². These assumptions may be motivated by considering the case in which simple random samples of participants may be taken from superpopulations, and completely randomized designs can be conducted for the participants. In this case, the (x₁, σ₁²), (x₂, σ₂²), . . . can be independent and identically distributed in the super-population(s).

### (1) Assumption 1

The predictors log(s₁²), log(s₂²), . . . for a super-population of participants are independent and identically distributed with finite mean and variance.

### (2) Assumption 2

The predictors vᵢ and the participant-level variances σᵢ² in the super-population are distributed such that, for any finite sample of size N>K+1 of participants from the superpopulation, the log{(1−hᵢᵢ)²σᵢ²+Σ_{k≠i}hᵢₖ²σₖ²} are identically distributed with finite mean and variance.

### (3) Assumption 3

For any finite sample of size N>K+1 of participants from the super-population, let F_{H}_{_{σ}}_{,N} denote the cumulative distribution function of the log{(1−hᵢᵢ)²σᵢ²+Σ_{k≠i}hᵢₖ²σₖ²}, . . . , log{(1−h_{NN})²σ_{N}²+Σ_{k≠N}h_{Nk}²σₖ²}. As N→∞, F_{H}_{_{σ}}_{,N} converges pointwise to a cumulative distribution function F_{H}_{_{σ}} with finite mean and variance.

To simplify the presentation of the results, systems implemented in accordance with certain embodiments of the invention may let log(S²) denote a random variable with the same distribution as the log(s₁²), log(s₂²), . . . in the super-population and log(H_{σ}) denote the logarithmic transformation of the random variable H_{σ} that has a cumulative distribution function F_{H}_{_{σ}}. Therefore, under the above three assumptions, as N→∞,

$$𝔼 ( {\hat{γ}}_{0} ) \to \frac{𝔼 [ {\{ log ( {S}^{2} ) \}}^{2} ] 𝔼 \{ log ( {H}_{σ} ) \} - 𝔼 \{ log ( {S}^{2} ) \} 𝔼 \{ log ( {S}^{2} ) log ( {H}_{σ} ) \}}{V a r \{ log ( {S}^{2} ) \}} - ( {γ}_{EM} + log 2 ) ,$$

$$𝔼 ( {\hat{γ}}_{1} ) \to \frac{Cov \{ log ( {S}^{2} ) , log ( {H}_{σ} ) \}}{Var \{ log ( {S}^{2} ) \}} ,$$

where γ_{EM}≈0.577 is the Euler-Mascheroni constant.

Systems and methods in accordance with various embodiments of the invention may ultimately conclude under the finite-sample and/or asymptotic perspectives that Weighted PROCOVA yields an unbiased treatment effect estimator.

### 3. Fitted Function Variance Reduction

In accordance with many embodiments of the invention, the power boost from Weighted PROCOVA may be considered a direct consequence of its reduction of the variance of the treatment effect estimator compared to PROCOVA. A description of the basis for this reduction of variance is disclosed in Romano, J. P. and Wolf, M. (2017). Resurrecting weighted least squares. Journal of Econometrics, 197(1):1-19, the disclosure of which, in particular the formulae of section 3, establishing the asymptotic distribution of \hat{β} under assumptions/conditions on the predictors and skedastic function, is incorporated by reference in its entirety. Systems in accordance with some embodiments of the invention may, in assessing variance reduction, assume that no residual from fitted PROCOVA models will ever be exactly equal to zero.

As disclosed above, in accordance with certain embodiments of the invention, values for the expected variance reduction in minimizing skedastic model and/or regression model coefficients may be obtained in multiple ways. Systems and methods in accordance with various embodiments of the invention may proceed under the assumption that \hat{β} converges to a Normal distribution centered at β. Additionally or alternatively, the asymptotic covariance matrix may be used to characterize the variance reduction of Weighted PROCOVA as a function of the predictors in the mean model, the limit of the fitted skedastic function model, and the participant-level variance.

Where [symbol] : [symbol] → [symbol] _{>0} denotes the limit of the fitted skedastic function model (that has as its input sᵢ²), in accordance with many embodiments of the invention, the asymptotic covariance matrix of \hat{β} under Weighted PROCOVA may take the form:

$${Ω}_{1 / 𝒢 ( {s}^{2} )}^{− 1} {Ω}_{{σ}^{2} / 𝒢 ( {s}^{2} )} {Ω}_{1 / 𝒢 ( {s}^{2} )}^{− 1} , \qquad ( 14 )$$

$$where$$

$${Ω}_{1 / 𝒢 ( {s}^{2} )} = E \{ \frac{1}{𝒢 ( {s}_{i}^{2} )} W \} , {Ω}_{{σ}^{2} / 𝒢 ( {s}^{2} )} = E \{ \frac{{σ}_{i}^{2}}{𝒢 {( {s}_{i}^{2} )}^{2}} W \} , W = ( \begin{matrix}1 & {w}_{i} & {m}_{i} \\ {w}_{i} & {w}_{i} & {w}_{i} {m}_{i} \\ {m}_{i} & {w}_{i} {m}_{i} & {m}_{i}^{2}\end{matrix} ) .$$

Additionally or alternatively, the asymptotic covariance matrix of \hat{β} under standard PROCOVA may take the form: [E{W}]⁻¹E{σᵢ²W}⁻¹[E{W}]⁻¹.

Systems in accordance with certain embodiments of the invention, may use the above asymptotic covariance matrices of \hat{β} to evaluate the variance reduction from using Weighted PROCOVA. In doing so, systems may assume that mᵢ, mᵢ², wᵢ, and wᵢmᵢ are uncorrelated with each of: the transformed predictors (1/ [symbol] (sᵢ²); the participant-level variances (σᵢ²); and the ratios of the participant-level variances to the square of the transformed predictors, (i.e., σᵢ²{ [symbol] (sᵢ²)}⁻²). Under the above assumptions, in accordance with several embodiments of the invention, the calculated variance reduction from using Weighted PROCOVA may take the form:

$$1 - \frac{𝔼 [ {σ}_{i}^{2} {\{ 𝒢 ( {s}_{i}^{2} ) \}}^{- 2} ]}{𝔼 \{ 𝒢 {( {s}_{i} )}^{- 2} \} 𝔼 ( {σ}_{i}^{2} )} \qquad ( 15 )$$

Additionally or alternatively, in the limiting case of [symbol] (sᵢ²)=exp{γ₀+γ₁ log(sᵢ²)}, the variance reduction may be a function of γ₁.

Systems and techniques directed towards weighted regression for the assessment of uncertainty, in accordance with many embodiments of the invention, are not limited to use within PROCOVA paradigms. Accordingly, it should be appreciated that applications described herein can be implemented in contexts including but not limited to various prognostic modelling configurations and in contexts unrelated to PROCOVA paradigms and/or medical trials, such as optimization in tellurene nanomanufacturing. Moreover, any of the systems and methods described herein with reference to FIGS. 4-7 can be utilized within any of the randomized experiment arrangements described above.

### III. Simulation Studies

By calculating the standard error of treatment effect estimator through methods including but not limited to bootstrap and/or sandwich estimators, systems implemented in accordance with several embodiments of the invention can conduct hypothesis tests that control the Type I error rate and create confidence intervals that have the desired coverage rate. These frequentist properties and applications (among others) are disclosed in the below scenarios.

In the below testing, two sets of simulation studies are used to compare and contrast the frequentist properties of Weighted PROCOVA with PROCOVA and the unadjusted analysis. The first set involves correctly specified mean and variance component models. The second set introduces a predictor in the data generation mechanism that is not in the statistical analysis to illuminate the impact of skedastic function model misspecification on the properties. In all of the simulations, assumptions include but are not limited to the assumption of no covariate drifts and/or changes in standard of care.

As mentioned above, treatment effect estimators in accordance with many embodiments of the invention may generate observed outcomes according to:

$${y}_{i} = {β}_{0} + {β}_{1} {w}_{i} + {β}_{2} {m}_{i} + {ϵ}_{i} \qquad ( 16 )$$

where ϵᵢ˜N(0, σᵢ²) independently, and the σᵢ² are generated according to:

$$log ( {σ}_{i}^{2} ) = {γ}_{0} + {γ}_{1} log ( {s}_{i}^{2} ) + {γ}_{2} {u}_{i , 2} + {ζ}_{i} \qquad ( 17 )$$

where u_{i,2} is an additional factor that can change the level of heteroskedasticity but is not included in any Weighted PROCOVA analysis, and ζᵢ˜N(0, ψ²) independently. The u_{i,2} values are introduced to evaluate the effects of skedastic function model misspecification on the frequentist properties of Weighted PROCOVA. For the first set of simulation studies, tests fix γ₂=0 to consider a correctly specified skedastic function model, and for the second set, tests consider a range of non-zero γ₂ values to consider a misspecified skedastic function model in terms of the omission of a predictor variable. The ζᵢ are unobservable sources of variation for the participant-level variances. Systems generate mᵢ˜N(0, τ₁²), log(sᵢ²)˜N(0, τ₂²), and u_{i,2}˜N(0, τ₂²) independently.

Each set of simulation studies involves three types of heteroskedasticity scenarios. The consideration of all these scenarios ultimately enables systems operating in accordance with miscellaneous embodiments of the invention to map out the statistical regimes for Weighted PROCOVA in terms of its frequentist properties.

In the first scenario, the total level of heteroskedasticity, defined as Var{log(σᵢ²)}, is fixed across all combinations of simulation parameters. The quantity Var{log(σᵢ²)} is the sum of the variation explained by the predictors of the skedastic function that are contained in xᵢ, and the variation due to unobserved sources. More formally, in this scenario γ₁²τ₂²+ψ² remains constant across the combinations of simulation parameters, or alternatively ψ=\sqrt{Var{log(σᵢ²)}−γ₁²τ₂²} for a fixed value of Var{log(σᵢ²)} and values of γ₁ and τ₂² such that γ₁²τ₂²<Var{log(σᵢ²)}. As log(sᵢ²) explains more of the variation in the log(σᵢ²) the variation of the ζᵢ decreases. This scenario enables characterization of the quality of log(sᵢ²) for Weighted PROCOVA in terms of the correlation between log(sᵢ²) and log(σᵢ²).

The second scenario differs from the first in that the participants' variances are a deterministic function of the predictors of the skedastic function, i.e., ψ=0 across combinations of simulation parameters. When γ₂=0 all variation in the log(σᵢ²) will arise from that of log(sᵢ²), and Var(log(σᵢ²)∝τ₂² with proportionality constant γ₁². When γ₁<1 then Var{log(σᵢ²)}<τ₂² and it may be expected that Weighted PROCOVA will not have desirable variance reduction or power boost properties, whereas when γ₁>1 then Var{log(σᵢ²)}>τ₂² and can be expected to perform well. Although the skedastic function is deterministic, study of the second scenario still enables systems implemented in accordance with several embodiments of the invention to learn how the change in the relationship between the log(sᵢ²) and log(σᵢ²) as characterized by γ₁ affects the frequentist properties of Weighted PROCOVA. This scenario was also considered by Romano, J. P. and Wolf, M. (2017). Resurrecting weighted least squares. Journal of Econometrics, 197(1): 1-19 (incorporated by reference in its entirety) and can arise in practice when subpopulations of participants with identical covariates have identical outcome variances.

The third and final scenario is more general than the first two in that the participants' variances are not a deterministic function of their covariates, and the total level of heteroskedasticity is not fixed across all combinations of simulation parameters. Specifically, ψ²>0 is fixed and Var{log(σᵢ²)} can change across the combinations of simulation parameters. This scenario helps understand how the combination of unobserved sources of variation and changes in the correlation between the log(sᵢ²) and log(σᵢ²) affect the performance of Weighted PROCOVA.

The analysis model generally follows the same mean model specification as in the formula for observed outcomes above. That said, in the below examples, the model includes log(sᵢ²) as the sole predictor in the skedastic function model and omits u_{i,2} from the analysis. Each setting has 10⁴ datasets simulated to evaluate the biases of \hat{β}₁, Type I error rates of the tests for H₀: \hat{β}₁=0, and coverage rates of the confidence intervals for β₁ for the three methods. For the power evaluations, tests set the true value of β₁ to 0.4, and for each combination of simulation parameters N is selected such that PROCOVA has 80% power for rejecting the null. The percentage variance reduction of Weighted PROCOVA may be evaluated compared to PROCOVA for the case of β₁=0, as additive treatment effects are considered throughout and the actual value of the treatment effect should not affect the variance reduction.

A simulation setting is defined by the combination of values for N, β₀, β₁, β₂, γ₀, γ₁, γ₂, τ₁², τ₂², τ₃², and ψ². Ranges of values may be considered for N, β₁, β₂, γ₀, γ₁, τ₂², and ψ². For the null case of β₁=0, the range of RCT sample sizes are N=50,100,300, 500, 1000. In all simulation settings the number of treated participants is N₁=N/2. Tests fix β₀=0 and τ₁²=τ₃²=1 throughout.

The values for γ₀, γ₁, γ₂, τ₂², and ψ² in the simulation studies are summarized in Table 1. The range of γ₁ values correspond to R² values for the skedastic function model fit being less than 0.1˜0.2 on average. Table 1 below discloses values of the parameters considered in the two sets of simulation studies. In the alternative case of β₁=0.4, the value of N depends on the other parameter values as it is selected so that PROCOVA has power 80% for rejecting the null. The first set of experiments has γ₂=0, and the second set has γ₂≠0.

TABLE 1

| Parameter | Setting 1 | Setting 2 | Setting 3 |
|---|---|---|---|
| N | 50, 100, 300, | 50, 100, 300, | 50, 100, 300, |
| (null case) | 500, 1000 | 500, 1000 | 500, 1000 |
| N | 250 | 210, 220, 240 | 350, 375, 400 |
| (alternative case) | | 250, 280, 325 | 425, 475, 525 |
| β₁ | 0, 0.4 | 0, 0.4 | 0, 0.4 |
| β₂ | 0.2, 0.3, 0.4, | 0.2, 0.3, 0.4, | 0.2, 0.3, 0.4, |
| | 0.5, 0.6 | 0.5, 0.6 | 0.5, 0.6 |
| γ₀ | −1.75 | 0 | 0 |
| γ₁ | 0.4, 0.6, | 0.4, 0.6, | 0.4, 0.6, |
| | 0.8, 1, 1.2, 1.4 | 0.8, 1, 1.2, 1.4 | 0.8, 1, 1.2, 1.4 |
| γ₂ | 0, 0.25, 0.5, | 0, 0.25, 0.5, | 0, 0.25, 0.5, |
| | 0.75, 1, | 0.75, 1, | 0.75, 1, |
| | 1.25, 1.5 | 1.25, 1.5 | 1.25, 1.5 |
| τ₂² | 0.5 | 0.5 | 0.5 |
| ψ² | 4 γ₁²τ₂² | 0 | 1 |

### A. Bias, Type I Error Rate, and Confidence Interval Coverage

Table 2 summarizes the average biases, Type I error rates, and confidence interval coverage rates for Weighted PROCOVA in the case of β₁=0 across the values of the other simulation parameters. Systems implemented in accordance with some embodiments may observe that Weighted PROCOVA yields unbiased estimators of the treatment effects, and controls the Type I error rates and confidence interval coverage rates. The Type I error rates, confidence interval coverages, and biases for Weighted PROCOVA, are averaged over the simulation parameter values in Table 1. The first set of simulation studies corresponds to the rows with γ₂=0, and the second set correspond to the rows with γ₂≠0.

TABLE 2

| | | Type I | Confidence | |
|---|---|---|---|---|
| | | Error | Interval | |
| Value of γ₂ | Setting | Rate | Coverage | Bias |
| γ₂ = 0 | Setting 1 | 0.0506 | 0.9494 | 10⁻⁴ |
| γ₂ = 0 | Setting 2 | 0.0558 | 0.9442 | 0 |
| γ₂ = 0 | Setting 3 | 0.0545 | 0.9455 | 0 |
| γ₂ ≠ 0 | Setting 1 | 0.0501 | 0.9499 | 0 |
| γ₂ ≠ 0 | Setting 2 | 0.0556 | 0.9444 | −10⁻⁴ |
| γ₂ ≠ 0 | Setting 3 | 0.0546 | 0.9454 | 10⁻⁴ |

Tables 3 and 4 contain inferences on the effects of the simulation parameters β₂, γ₁, γ₂, and N on these metrics. Specifically, systems may provide both the estimated main effects of these parameters and the p-values for testing whether they have any effects on the metrics. These inferences are obtained via linear regression on the results from the simulation studies. The tests show that all of the main effects for these parameters are estimated to be zero, and that none of them are statistically significant. This helps to indicate that for these ranges of (β₂, γ₁,γ₂, and N, these parameters don't affect the frequentist properties of Weighted PROCOVA.

Table 3 illustrates the main effects and p-values of the effects of the parameters β₂, γ₁, and N on the Type I error rates, confidence interval coverage rates, and biases of Weighted PROCOVA in the case of γ₂=0.

TABLE 3

| | | Main Effect (p-value) | Main Effect (p-value) | Main Effect (p-value) |
|---|---|---|---|---|
| Parameter | Setting | Type I Error Rate | Coverage Rate | Bias |
| βₘ | Setting 1 | −0.001 (0.394) | 0.001 (0.394) | −0.001 (0.5) |
| γ₁ | Setting 1 | 0.004 (<0.0001) | −0.004 (<0.0001) | 0 (0.87) |
| N | Setting 1 | 0 (0.235) | 0 (0.235) | 0 (0.884) |
| βₘ | Setting 2 | 0.002 (0.379) | −0.002 (0.379) | 0 (0.671) |
| γ₁ | Setting 2 | 0.003 (<0.0001) | −0.003 (<0.0001) | 0 (0.848) |
| N | Setting 2 | 0 (<0.0001) | 0 (<0.0001) | 0 (0.975) |
| βₘ | Setting 3 | −0.002 (0.36) | 0.002 (0.36) | 0 (0.991) |
| γ₁ | Setting 3 | 0.003 (<0.0001) | −0.003 (<0.0001) | 0 (0.864) |
| N | Setting 3 | 0 (<0.0001) | 0 (<0.0001) | 0 (0.842) |

Table 4: Main effects and p-values of the effects of the parameters β₂, γ₁, γ₂, and N on the Type I error rates, confidence interval coverage rates, and biases of Weighted PROCOVA in the case of γ₂≠0.

TABLE 4

| | | Main Effect (p-value) | Main Effect (p-value) | Main Effect (p-value) |
|---|---|---|---|---|
| Parameter | Setting | Type I Error Rate | Coverage Rate | Bias |
| γ₂ | Setting 1 | −0.003 (<0.0001) | 0.003 (<0.0001) | −0.001 (0.259) |
| γ₁ | Setting 1 | 0.004 (<0.0001) | −0.004 (<0.0001) | 0 (0.403) |
| N | Setting 1 | 0 (0.02) | 0 (0.02) | 0 (0.547) |
| γ₂ | Setting 2 | −0.001 (0.279) | 0.001 (0.279) | 0 (0.774) |
| γ₁ | Setting 2 | 0.003 (0.005) | −0.003 (0.005) | −0.001 (0.031) |
| N | Setting 2 | 0 (<0.0001) | 0 (<0.0001) | 0 (0.281) |
| γ₂ | Setting 3 | −0.002 (0.016) | 0.002 (0.016) | 0 (0.86) |
| γ₁ | Setting 3 | 0.003 (0.003) | −0.003 (0.003) | 0 (0.503) |
| N | Setting 3 | 0 (<0.0001) | 0 (<0.0001) | 0 (0.476) |

### B. Variance Reduction and Power Boost

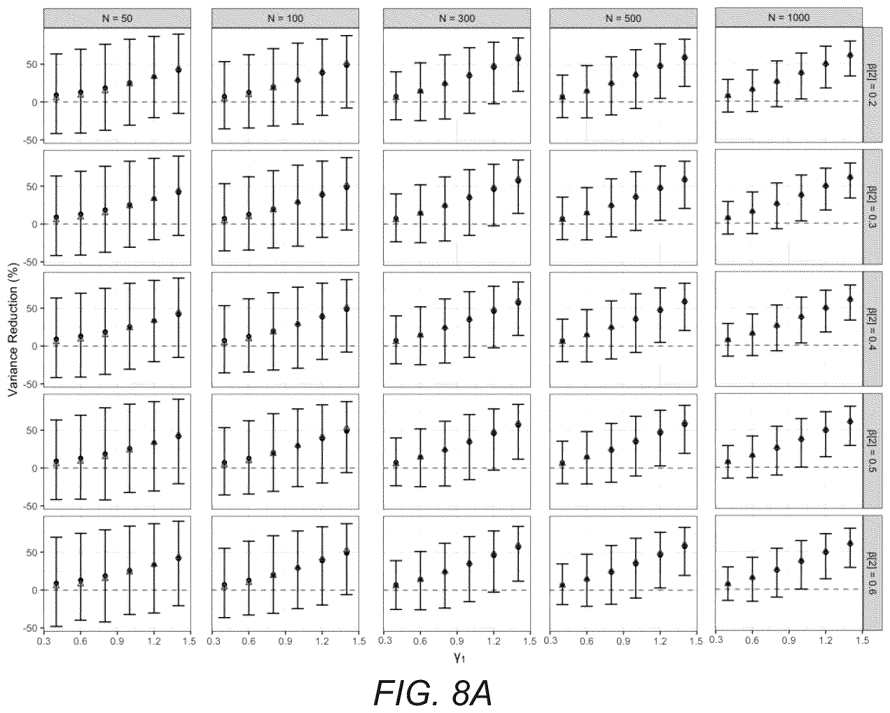

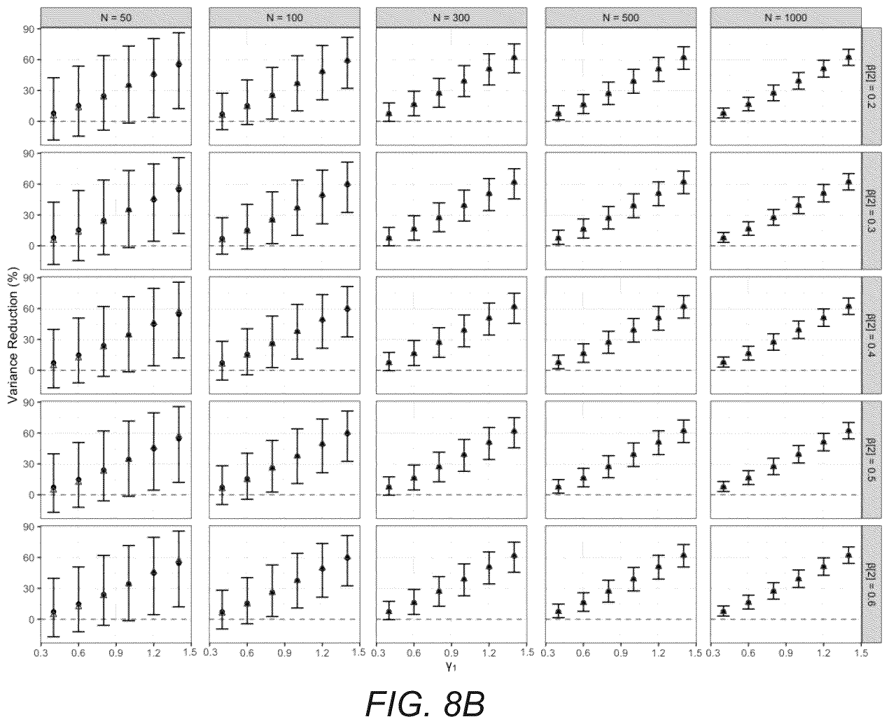

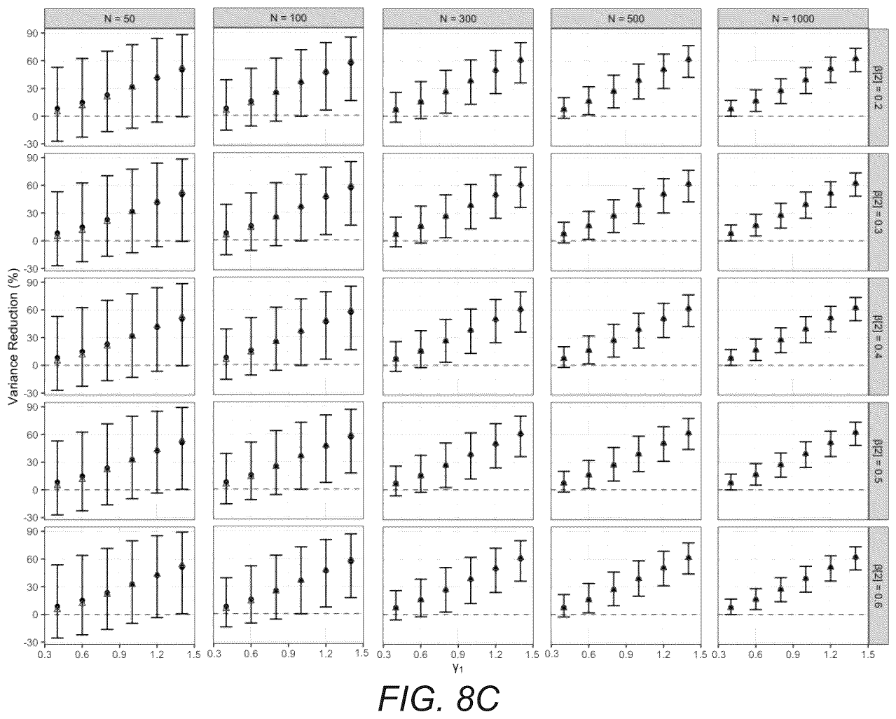

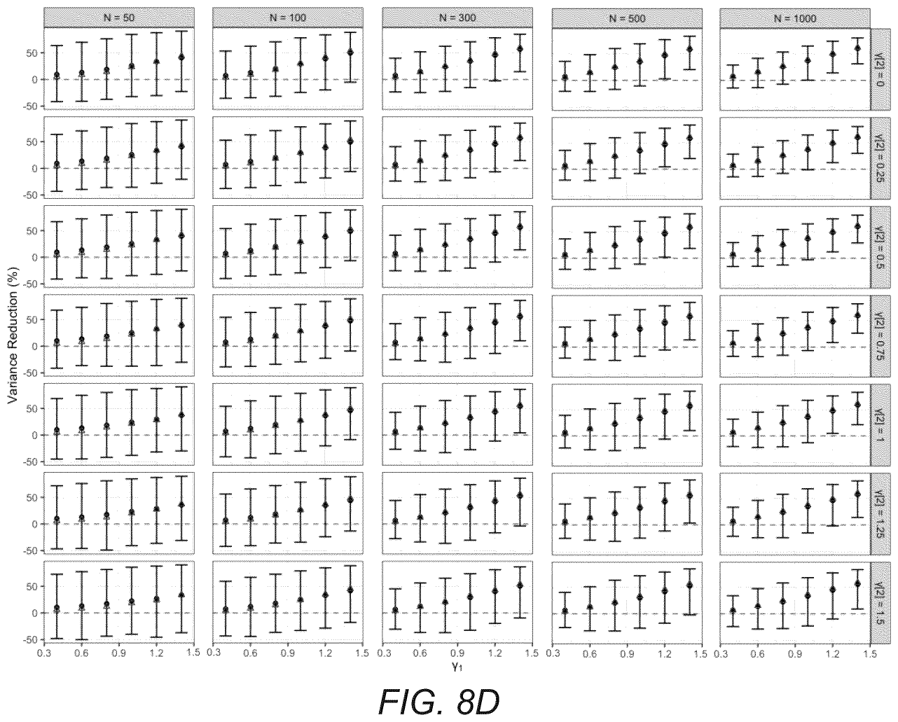

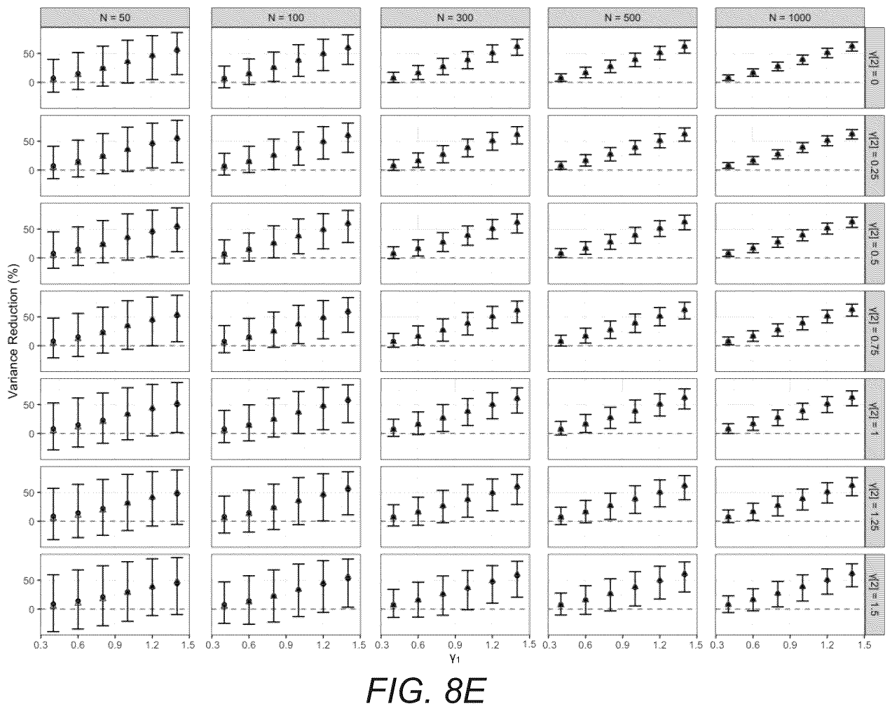

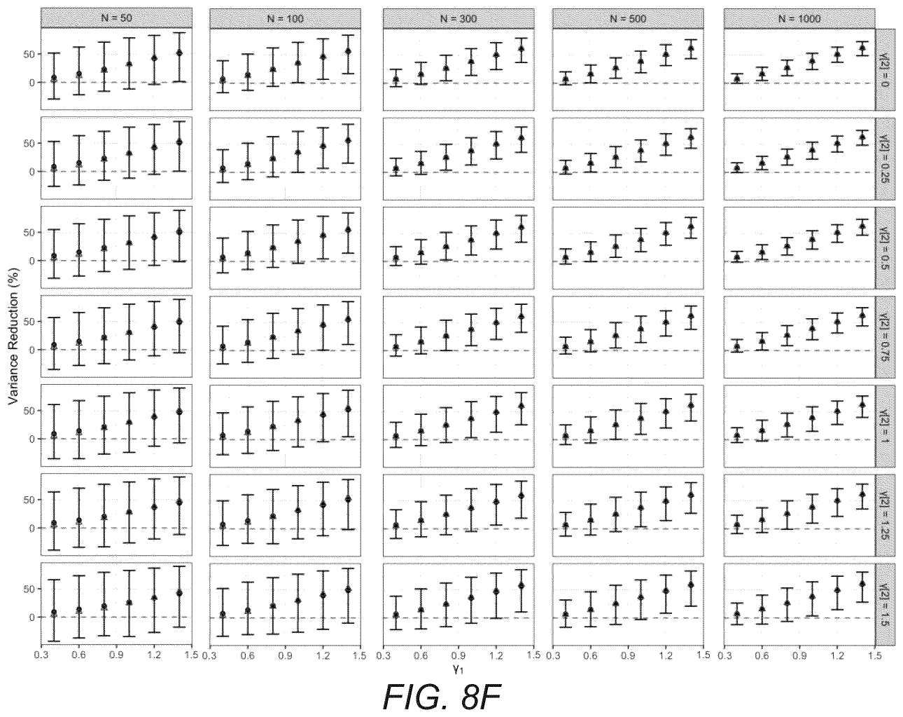

FIGS. 8A to 8F summarize percentage variance reductions of Weighted PROCOVA, implemented in accordance with several embodiments of the invention, when measured compared to PROCOVA. These variance reductions are measured for two sets of simulations (in which γ₂=0 and γ₂≠0, where β₁=0 throughout) and the three heteroskedasticity scenarios.
FIG. 8A depicts the variance reduction for the first heteroskedasticity scenario when γ₂=0;
FIG. 8B depicts the variance reduction for the second heteroskedasticity scenario when γ₂=0;
FIG. 8C depicts the variance reduction for the third heteroskedasticity scenario when γ₂=0;
FIG. 8D depicts the variance reduction for the first heteroskedasticity scenario when γ₂≠0;
FIG. 8E depicts the variance reduction for the second heteroskedasticity scenario when γ₂≠0; and
FIG. 8F depicts the variance reduction for the third heteroskedasticity scenario when γ₂≠0.
   In each of the figures, the triangle indicates the median of the percentage variance reduction, and the dot indicates the mean of the percentage variance reduction, across the simulated datasets. As suggested in the figures, variance reduction is significantly affected by γ₁, and increases as a function of γ₁. As N increases, the variation in variance reduction decreases, and the probability of an inflation of the variance compared to PROCOVA decreases. In addition, as γ₂ increases, the variation in the variance reduction increases, and the probability of an inflation of the variance compared to PROCOVA increases. The N and γ₂ parameters seem to have an interaction effect on variance reduction (FIGS. 8D to 8F). The values of β₂ do not seem to affect variance reduction. Lastly, the second and third heteroskedasticity scenarios exhibit less variation in variance reduction compared to the first scenario for the cases of γ₂=0 and γ₂≠0, respectively.

The dependence of Weighted PROCOVA's performance metrics on γ₁ corresponds to the fact that, as γ₁ increases, the sᵢ² values explain more of the heteroskedasticity in the outcomes. This also follows from the fact that γ₁ is related to the expected coefficient of determination R² value for the skedastic function model fit. The values of γ₁ in the simulation studies correspond to the expected R² values ranging from 0.1 to 0.2 for the skedastic function model fit. The percentage of variance reduction offered by Weighted PROCOVA can reach up to 50%, even when the skedastic function model is incorrectly specified, so long as the variance explains the heteroskedasticity in the RCT. For small γ₁ the amount of heteroskedasticity would be negligible, so it may be preferable to use PROCOVA.

Simulations implemented in accordance with various embodiments of the invention are not limited to use alongside Weighted PROCOVA. Accordingly, it should be appreciated that applications described herein may also be implemented outside the context of PROCOVA methodology. Moreover, while specific simulation studies are described in the figures and tables above, a variety of simulation configurations can be utilized to evaluate covariate adjustments as appropriate to the requirements of specific applications.

### IV. Systems for Determining Treatment Effects

### A. Treatment Analysis System

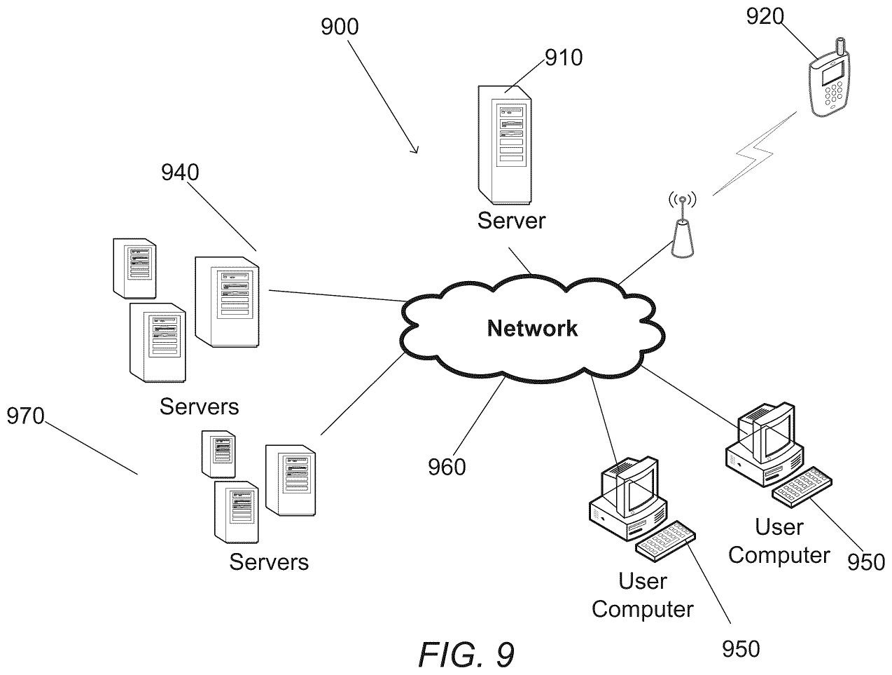

An example of a treatment analysis system that determines treatment effects in accordance with some embodiments of the invention is illustrated in FIG. 9. Network 900 includes a communications network 960. The communications network 960 is a network such as the Internet that allows devices connected to the network 960 to communicate with other connected devices. Server systems 910, 940, and 970 are connected to the network 960. Each of the server systems 910, 940, and 970 is a group of one or more servers communicatively connected to one another via internal networks that execute processes that provide cloud services to users over the network 960. One skilled in the art will recognize that a treatment analysis system may exclude certain components and/or include other components that are omitted for brevity without departing from this invention.

For purposes of this discussion, cloud services are one or more applications that are executed by one or more server systems to provide data and/or executable applications to devices over a network. The server systems 910, 940, and 970 are shown each having three servers in the internal network. However, the server systems 910, 940 and 970 may include any number of servers and any additional number of server systems may be connected to the network 960 to provide cloud services. In accordance with various embodiments of this invention, treatment analysis systems in accordance with various embodiments of the invention may be provided by a process being executed on a single server system and/or a group of server systems communicating over network 960.

Users may use personal devices 980 and 920 that connect to the network 960 to perform processes that determine treatment effects in accordance with various embodiments of the invention. In the shown embodiment, the personal devices 980 are shown as desktop computers that are connected via a conventional “wired” connection to the network 960. However, the personal device 980 may be a desktop computer, a laptop computer, a smart television, an entertainment gaming console, and/or any other device that connects to the network 960 via a “wired” connection. The mobile device 920 connects to network 960 using a wireless connection. A wireless connection is a connection that uses Radio Frequency (RF) signals, Infrared signals, and/or any other form of wireless signaling to connect to the network 960. In FIG. 9, the mobile device 920 is a mobile telephone. However, mobile devices 920 may be mobile phones, Personal Digital Assistants (PDAs), tablets, smartphones, and/or any other type of device that connects to network 960 via wireless connection without departing from this invention.

As can readily be appreciated the specific computing system used to determine treatment effects is largely dependent upon the requirements of a given application and should not be considered as limited to any specific computing system(s) implementation.

### B. Treatment Analysis Element

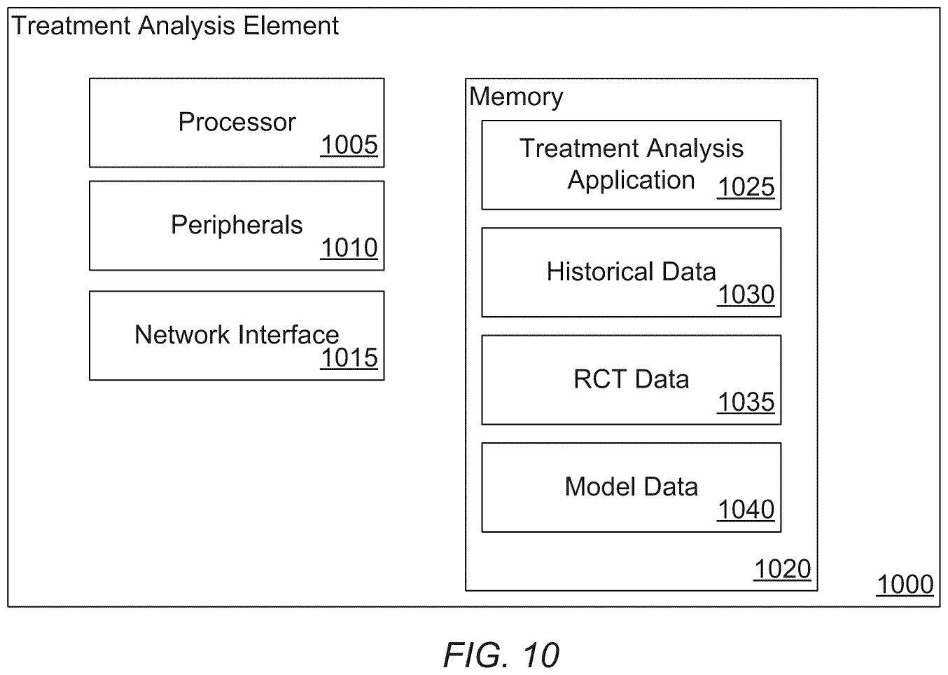

An example of a treatment analysis element that executes instructions to perform processes that determine treatment effects in accordance with various embodiments of the invention is illustrated in FIG. 10. Treatment analysis elements in accordance with many embodiments of the invention can include (but are not limited to) one or more of mobile devices, cloud services, and/or computers. Treatment analysis element 1000 includes processor 1005, peripherals 1010, network interface 1015, and memory 1020. One skilled in the art will recognize that a treatment analysis element may exclude certain components and/or include other components that are omitted for brevity without departing from this invention.

The processor(s) 1005 can include (but is not limited to) a processor, microprocessor, controller, and/or a combination of processors, microprocessor, and/or controllers that perform instructions stored in the memory 1020 to manipulate data stored in the memory. Processor instructions can configure processors 1005 to perform processes in accordance with certain embodiments of the invention.

Peripherals 1010 can include any of a variety of components for capturing data, such as (but not limited to) cameras, displays, and/or sensors. In a variety of embodiments, peripherals can be used to gather inputs and/or provide outputs. Treatment analysis element 1000 can utilize network interface 1015 to transmit and receive data over a network based upon the instructions performed by processor 1005. Peripherals 1010 and/or network interfaces 1015 in accordance with many embodiments of the invention can be used to gather data that can be used to determine treatment effects.

Memory 1020 includes a treatment analysis application 1025, historical data 1030, RCT data 1035, and model data 1040. Treatment analysis applications in accordance with several embodiments of the invention can be used to determine treatment effects of an RCT, to design an RCT, and/or determine decision rules for treatments.

Historical data 1030 in accordance with many embodiments of the invention can be used to pre-train generative models to generate potential outcomes for digital subjects and/or digital twins. In numerous embodiments, historical data 1030 can include (but is not limited to) control arms from historical control arms, patient registries, electronic health records, and/or real-world data. In many embodiments, predictions from generative models can be compared to historical data 1030 that was not used to train the models in order to obtain prior distributions capturing how well the predictions generalize to new populations.

In some embodiments, RCT data 1035 can include panel data collected from subjects of RCTs. RCT data 1035 in accordance with a variety of embodiments of the invention can be divided into control and treatment arms based on whether subjects received a treatment. In many embodiments, RCT data 1035 can be supplemented with generated subject data. Generated subject data in accordance with a number of embodiments of the invention can include (but is not limited to) digital subject data and/or digital twin data.

In several embodiments, model data 1040 can store various parameters and/or weights for generative models. Model data 1040 in accordance with many embodiments of the invention can include data for models trained on historical data 1030 and/or trained on RCT data 1035. In several embodiments, pre-trained models can be updated based on RCT data 1035 to generate digital subjects.

Although a specific example of a treatment analysis element 1000 is illustrated in this figure, any of a variety of treatment analysis elements can be utilized to perform processes for determining treatment effects similar to those described herein as appropriate to the requirements of specific applications in accordance with embodiments of the invention.

### C. Treatment Analysis Application

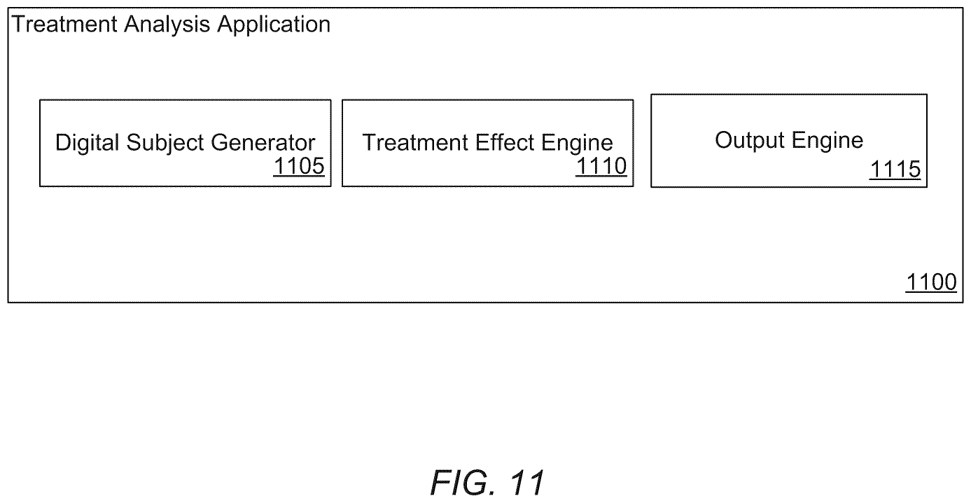

An example of a treatment analysis application for determining treatment effects in accordance with some embodiments of the invention is illustrated in FIG. 11. Treatment analysis applications 1100 includes digital subject generator 1105, treatment effect engine 1110, and output engine 1115. One skilled in the art will recognize that a treatment analysis application may exclude certain components and/or include other components that are omitted for brevity without departing from this invention.

Digital subject generators 1105 in accordance with various embodiments of the invention can include generative models that can generate digital subject and/or digital twin data. Generative models in accordance with certain embodiments of the invention can be trained to generate potential outcome data based on characteristics of an individual and/or a population. Digital subject data in accordance with several embodiments of the invention can include (but is not limited to) panel data, outcome data, etc. In several embodiments, generative models can include (but are not limited to) traditional statistical models, generative adversarial networks, recurrent neural networks, Gaussian processes, autoencoders, autoregressive models, variational autoencoders, and/or other types of probabilistic generative models.

In various embodiments, treatment effect engines 1110 can be used to determine treatment effects based on generated digital subject data and/or data from a RCT. In some embodiments, treatment effect engines 1110 can use digital subject data from digital subject generators 1105 to determine treatment effects in a variety of different applications, such as, but not limited to, comparing separate generative models based on data from the control and treatment arms of RCTs, supplementing control arms in RCTs, comparing predicted potential control outcomes with actual treatment outcomes, etc. Treatment effect engines 1110 in accordance with many embodiments of the invention can be used to determine individualized responses to treatment. In certain embodiments, treatment effect engines 1110 can determine biases of generative models of the digital subject generator and incorporate the biases (or corrections for the biases) in the treatment effect analyses.

Output engines 1115 in accordance with several embodiments of the invention can provide a variety of outputs to a user, including (but not limited to) decision rules, treatment effects, generative model biases, recommended RCT designs, etc. In numerous embodiments, output engines 1115 can provide feedback when the results of generative models of a digital subject generator 1105 diverge from the RCT population. For example, output engines in accordance with certain embodiments of the invention can provide a notification when a difference between generated control outcomes for digital twins of subjects from a control arm and their actual control outcomes exceeds a threshold.

Although a specific example of a treatment analysis application is illustrated in this figure, any of a variety of Treatment analysis applications can be utilized to perform processes for determining treatment effects similar to those described herein as appropriate to the requirements of specific applications in accordance with embodiments of the invention.

Systems and techniques for applying prognostic models to assessment of experiment uncertainty, are not limited to use for randomized controlled trials. Accordingly, it should be appreciated that applications described herein can be implemented outside the context of generative model architecture and in contexts unrelated to RCTs. Moreover, any of the systems and methods described herein with reference to FIGS. 1-11 can be utilized within any of the generative models described above.

Although specific methods of determining treatment effects are discussed above, many different methods of treatment analysis can be implemented in accordance with many different embodiments of the invention. It is therefore to be understood that the present invention may be practiced in ways other than specifically described, without departing from the scope and spirit of the present invention. Thus, embodiments of the present invention should be considered in all respects as illustrative and not restrictive. Accordingly, the scope of the invention should be determined not by the embodiments illustrated, but by the appended claims and their equivalents.

What is claimed is:

1. A method for estimating treatment effects for a target trial, the method comprising:
defining a skedastic function model, wherein defining the skedastic function model depends, at least in part, on trial data that was applied in a trial;
designing trial parameters for a target trial based in part on the skedastic function model;
applying the trial parameters to a loss function to derive at least one minimizing outcome coefficient, wherein the at least one minimizing outcome coefficient corresponds to a regression coefficient for an expected outcome to the target trial based on the trial parameters;
computing standard errors for the at least one minimizing outcome coefficient;
quantifying, using the standard errors, values for uncertainty associated with the target trial; and
updating the trial parameters according to the uncertainty.

2. The method of claim 1, wherein the standard errors are heteroskedasticity-consistent standard errors.

3. The method of claim 1, wherein the expected outcome is obtained through at least one of the group consisting of a digital twin and a prognostic model.

4. The method of claim 1, wherein defining the skedastic function model comprises:
calculating one or more predicted outcomes for the trial data;
obtaining residuals corresponding to the one or more predicted outcomes for the trial data; and
using the residuals to define the skedastic function model.

5. The method of claim 4, wherein predicted outcomes for the trial data are based on digital twin outputs.

6. The method of claim 5, wherein:
the predicted outcomes are predictions from a regression model fitted on the trial data; and
predictors of the regression model are means of the digital twin outputs.

7. The method of claim 4, wherein the trial data comprises participant data for an RCT.

8. The method of claim 4, wherein defining the skedastic function model further comprises:
applying parameters of the skedastic function model to a loss function for data from the target trial, to derive at least one minimizing model coefficient, wherein the at least one minimizing model coefficient includes a treatment effect coefficient;
computing standard errors for the at least one minimizing model coefficient;
calculating one or more predicted outcomes for the target trial; and
defining the skedastic function model further based on variances corresponding to the one or more predicted outcomes for the target trial.

9. The method of claim 8, wherein:
predicted outcomes for the target trial are based on digital twin outputs; and
minimizing model coefficients are treatment effect coefficients.

10. The method of claim 1, wherein the loss function is a weighted least squares loss function.

11. The method of claim 10, wherein at least one weight quantity of the weighted least squares loss function is inversely proportional to a predicted variance of outcomes of a participant in the target trial.

12. The method of claim 10, wherein each weight quantity of the weighted least squares loss function has a positive value.

13. The method of claim 10, wherein at least one weight quantity of the weighted least squares loss function is defined by:
implementing, using trial data, an ordinary least squares fit;
obtaining least squares coefficients from the ordinary least squares fit; and
deriving, from the least squares coefficients and the trial parameters, the at least one weight quantity.

14. The method of claim 1, wherein:
updating the trial parameters according to the uncertainty comprises determining a set of characteristics for the target trial, wherein the set of characteristics comprises a number of subjects to be enrolled in each of a control arm and a treatment arm; and
the uncertainty is based on at least one of a desired type-I error rate and a desired type-II error rate.

15. The method of claim 1, wherein updating the trial parameters comprises at least one of:
minimizing a total number of samples for at least one selected from the group consisting of a treatment arm of the target trial, a control arm of the target trial, and the target trial in totality; and
performing a regression analysis based on the expected outcome.

16. The method of claim 15, wherein:
an estimate for coefficients of the regression analysis is represented as:
$$\hat{β} = {( {Z}^{T} Z )}^{- 1} {Z}^{T} Y$$
Y is a vector corresponding to treatment outputs for each participant; and
Z is a matrix for which each row (zᵢ) corresponds to a set of predictor variables for a participant (i).

17. The method of claim 16, wherein the set of predictor variables for each participant comprise the expected outcome and a corresponding treatment for the participant.

18. The method of claim 15, wherein minimizing a total number of samples is performed by deriving an expected variance reduction.

19. The method of claim 18, wherein deriving the expected variance reduction comprises:
obtaining a limit for the skedastic function model;
deriving a set of estimated variance reductions for the previous trial, wherein the estimated variance reduction for each participant of the previous trial is derived from a ratio between a diagonal entry of a first matrix and a diagonal entry of a second matrix; and
determining the expected variance reduction from the set of estimated variance reductions.

20. The method of claim 19, wherein:
Xᵢ is a vector of predictor variables for a participant i;
sᵢ² is a representation of the unknown outcome variance for the participant i;
the first matrix is represented as:
$${Ω}_{1 / 𝒢 ( {σ}_{i}^{2} )}^{- 1} {Ω}_{{s}^{2} / 𝒢 {( {σ}_{i}^{2} )}^{2}} {Ω}_{1 / 𝒢 ( {σ}_{i}^{2} )}^{- 1} , where :$$
$${Ω}_{1 / 𝒢 ( {σ}_{i}^{2} )} = E ( \frac{1}{𝒢 ( {σ}_{i}^{2} )} {X}_{i} {X}_{i}^{T} ) ,$$
$${Ω}_{{s}^{2} / 𝒢 {( {σ}_{i}^{2} )}^{2}} = E ( \frac{{s}_{i}^{2}}{{𝒢 ( {σ}_{i}^{2} )}^{2}} {X}_{i} {X}_{i}^{T} ) ,$$
and [symbol]
(σᵢ²) is the limit of the skedastic function model for the participant i; and
the second matrix is represented as: Ω⁻¹Ωₛ_{²}Ω⁻¹, where:
Ω=E(XᵢXᵢ^{T}), and
Ωₛ_{²}=E(sᵢ²XᵢXᵢ^{T}).
